(19) United States
(12) Patent Application Publication
(10) Pub. No.: US 2004/0064901 A1
(43) Pub. Date: Apr. 8, 2004

(54) Enzymatic coloring agents

(76) Inventors: Astrid Kleen, Erkrath (DE); Bianca Frauendorf, Velbert (DE)

Correspondence Address: HENKEL CORPORATION, THE TRIAD, SUITE 200, 2200 RENAISSANCE BLVD., GULPH MILLS, PA, 19406, US

(21) Appl. No.: 10/432,243

(22) PCT Filed: Nov. 10, 2001
(86) PCT No.: PCT/EP01/13025

(30) Foreign Application Priority Data
Nov. 20, 2000 (DE) 100 57 547.1
Apr. 4, 2001 (DE) 101 16 759.8

(51) Int. Cl. A61K 7/13

(52) U.S. Cl. 8/405

(57) ABSTRACT

The invention relates to agents for coloring keratin fibers which, in a cosmetically acceptable carrier, contains: (A) at least one colorant precursor; (B) at least one enzyme, which can catalyze the oxidation of the colorant precursors; (C) at least one sugar surfactant selected from the group that is formed from alkyl oligoglycosides, alkenyl oligoglycosides and from fatty acid N-alkyl-polyhydroxyalkylamides, and; (D) at least one reducing agent. The inventive agents are characterized by their good ability to color and by their action that is gentle on hair.

This invention relates to preparations for coloring keratin fibers which contain inter alia at least one enzyme capable of catalyzing the oxidation of dye precursors and an active-substance combination of at least one sugar surfactant and a reducing agent, to the use of these preparations for coloring keratin fibers and to various processes for coloring keratin fibers with these preparations.

Nowadays, human hair is treated in many different ways with haircare preparations. Such treatments include, for example, the cleaning of hair with shampoos, the care and regeneration of hair with rinses and treatments and the bleaching, coloring and shaping of hair with coloring and tinting formulations, wave formulations and styling preparations. Among these, formulations for modifying or shading the color of the hair occupy a prominent position.

Colorants or tints containing substantive dyes as their coloring component are normally used for temporary colors. Substantive dyes are based on dye molecules which are directly absorbed onto the hair and do not require an oxidative process for developing the color. Dyes such as these include, for example, henna which has been used since ancient times for coloring the body and hair. Corresponding colors are generally much more sensitive to shampooing than oxidative colors so that an often unwanted change of shade or even a visible “decoloration” can occur very much more quickly.

So-called oxidation colorants are used for permanent, intensive colors with corresponding fastness properties. Oxidation colorants normally contain oxidation dye precursors, so-called primary intermediates and secondary intermediates. The primary intermediates form the actual dyes with one another or by coupling with one or more secondary intermediates under the influence of oxidizing agents or atmospheric oxygen. Oxidation colorants are distinguished by excellent long-lasting coloring results. Natural-looking colors normally require a mixture of a relatively large number of oxidation dye precursors; in many cases, substantive dyes are used for shading.

Basically, oxidative development of the color may be carried out with atmospheric oxygen although a chemical oxidizing agent is preferably used. Suitable oxidizing agents are persulfates, chlorites and, in particular, hydrogen peroxide or addition products thereof with urea, melamine and also sodium borate. A ca. 2-9% aqueous hydrogen peroxide solution is normally used. Concentrations of oxidizing agent as high as these can damage the keratinous fibers, particularly where they are already “permed” or bleached, and on occasion can even cause irritation of the skin.

A major contribution towards solving this problem is based on reducing the concentration of oxidizing agent. Accordingly, attempts have been made in the past on the one hand to find dye precursors which, on the basis of their chemical structure, can be oxidized even by small quantities of hydrogen peroxide or by atmospheric oxygen. On the other hand, it has been proposed to use enzymes as biocatalysts because they are capable of catalyzing the required oxidation process with very little, if any, hydrogen peroxide solely in the presence of atmospheric oxygen.

DE-OS 21 55 390 describes an enzyme-activated oxidative hair coloring process in which small quantities of H₂O₂ are used in combination with a peroxidase. EP-A1 0 310 675 also discloses enzymatic hair treatment preparations containing at least one two-electron-reducing oxidase which uses oxygen as acceptor. EP-B1-0 548 620 describes enzymatic hair colorants where oxidation of the dye precursors is catalyzed by a peroxidase.

Finally, EP-A2-0 795 313 describes enzymatic hair colorants containing an oxygen-oxidoreductase/substrate system and a peroxidase and, as secondary intermediate, an m-phenylenediamine derivative which is compulsory. However, none of these colorants is totally convincing in terms of coloring performance (intensity, shade, luster, fastness properties).

The art of readily oxidizable dye precursors, like the enzymatic development of colors described hitherto, has the disadvantage that the results obtained are poorer by comparison with conventional processes, particularly in regard to the intensity, luster and fastness properties of the colors. In addition, known enzymatic hair colorants are attended by the disadvantage that relatively large quantities of enzymes are required to obtain standard coloring results.

Accordingly, the problem addressed by the present invention was to provide enzymatic colorants for keratinous fibers which would fulfil the requirements colorants are expected to meet to a high degree, even where minimal quantities of enzyme and/or dye are used. Another problem addressed by the invention was to provide colorants which would be gentle on the skin and hair.

In a first embodiment, therefore, the present invention relates to preparations for coloring keratinous fibers which contain

(A) at least one dye precursor,

(B) at least one enzyme capable of catalyzing the oxidation of the dye precursors,

(C) at least one sugar surfactant selected from the group consisting of

alkyl and alkenyl oligoglycosides and

fatty acid-N-alkyl polyhydroxyalkyl amides and

(D) at least one reducing agent

in a cosmetically acceptable carrier.

In the context of the invention, keratinous fibers are understood to be pelts, wool, feathers and, in particular, human hair.

Dye Precursors

The invention is not limited in any way in regard to the dye precursors (A) used in the colorants according to the invention. The colorants according to the invention may contain

oxidation dye precursors of the primary intermediate and/or secondary intermediate type and

precursors of “nature-analogous” dyes, such as indole and indoline derivatives,

and mixtures of representatives of these groups as dye precursors.

In a first embodiment, the colorant contains at least one primary intermediate. The primary intermediates normally used are primary aromatic amines containing another free or substituted hydroxy or amino group in the para or ortho position, diaminopyridine derivatives, heterocyclic hydrazones, 4-aminopyrazolone derivatives and 2,4,5,6-tetraaminopyrimidine and derivatives thereof.

In a preferred embodiment of the invention, a p-phenylenediamine derivative or a physiologically compatible salt thereof is used as the primary intermediate. Particularly preferred p-phenylenediamine derivatives correspond to formula (E1):

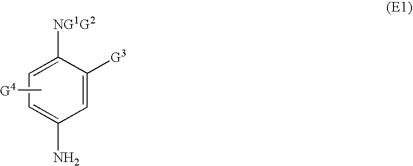

in which

G¹ stands for a hydrogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical, a 4′-aminophenyl radical or a C₁₋₄ alkyl radical substituted by a nitrogen-containing group, a phenyl group or a 4′-aminophenyl group;

G² stands for a hydrogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical or a C₁₋₄ alkyl radical substituted by a nitrogen-containing group;

G³ stands for a hydrogen atom, a halogen atom, such as a chlorine, bromine, iodine or fluorine atom, a C₁₋₄ alkyl radical, a C₁₋₄ monohydroxyalkyl radical, a C₁₋₄ hydroxyalkoxy radical, a C₁₋₄ acetylaminoalkoxy radical, a C₁₋₄ mesylaminoalkoxy radical or a C₁₋₄ carbamoylaminoalkoxy radical;

G⁴ is a hydrogen atom, a halogen atom or a C₁₋₄ alkyl radical or

if G³ and G⁴ are in the ortho position to one another, they may together form a bridging α,ω-alkylenedioxo group such as, for example, an ethylenedioxy group.

Examples of the C₁₋₄ alkyl radicals mentioned as substituents in the compounds according to the invention are the methyl, ethyl, propyl, isopropyl and butyl groups. Ethyl and methyl radicals are preferred alkyl radicals. According to the invention, preferred C₁₋₄ alkoxy radicals are, for example, methoxy or ethoxy radicals. Other preferred examples of a C₁₋₄ hydroxyalkyl group are the hydroxymethyl, 2-hydroxyethyl, 3-hydroxypropyl or 4-hydroxybutyl group. A 2-hydroxyethyl group is particularly preferred. According to the invention, examples of halogen atoms are F, Cl or Br atoms. Cl atoms are most particularly preferred. According to the invention, the other terms used are derived from the definitions given here. Examples of nitrogen-containing groups corresponding to formula (E1) are, in particular, the amino groups, C₁₋₄ monoalkylamino groups, C₁₋₄ dialkylamino groups, C₁₋₄ trialkylammonium groups, C₁₋₄ monohydroxyalkylamino groups, imidazolinium and ammonium.

Particularly preferred p-phenylenediamines corresponding to formula (E1) are selected from p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)-aniline, N,N-bis-(β-hydroxyethyl)-p-phenylenediamine, 4-N,N-bis-(β-hydroxyethyl)-amino-2-methylaniline, 4-N,N-bis-(β-hydroxyethyl)-amino-2-chloroaniline, 2-(β-hydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl-β-hydroxyethyl)-p-phenylenediamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylenediamine and 5,8-diaminobenzo-1,4-dioxane and physiologically compatible salts thereof.

According to the invention, most particularly preferred p-phenylenediamine derivatives corresponding to formula (E1) are p-phenylenediamine, p-toluylenediamine, 2-(β-hydroxyethyl)-p-phenylenediamine and N,N-bis-(2-hydroxyethyl)-p-phenylenediamine.

In another preferred embodiment of the invention, compounds containing at least two aromatic nuclei substituted by amino and/or hydroxyl groups may be used as the primary intermediate.

The binuclear primary intermediates which may be used in the coloring compositions according to the invention include in particular compounds corresponding to formula (E2) and physiologically compatible salts thereof:

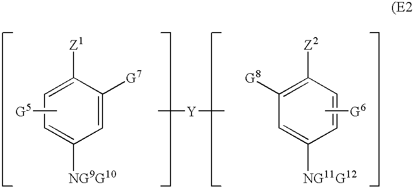

in which

Z¹ and Z² independently of one another stand for a hydroxyl or NH₂ radical optionally substituted by a C₁₋₄ alkyl radical, by a C₁₋₄ hydroxyalkyl radical and/or by a bridging group Y,

the bridging group Y is a C₁₋₁₄ alkylene group such as, for example, a linear or branched alkylene chain or an alkylene ring which may be interrupted or terminated by one or more nitrogen-containing groups and/or one or more hetero atoms, such as oxygen, sulfur or nitrogen atoms, and may optionally be substituted by one or more hydroxyl or C₁₋₈ alkoxy radicals,

G⁵ and G⁶ independently of one another stand for a hydrogen or halogen atom, a C₁₋₄ alkyl radical, a C₁₋₄ monohydroxyalkyl radical, a C₂₋₄ polyhydroxyalkyl radical, a C₁₋₄ aminoalkyl radical or a direct bond to the bridging group Y,

G⁷, G⁸, G⁹, G¹⁰, G¹¹ and G¹² independently of one another stand for a hydrogen atom, a direct bond to the bridging group Y or a C₁₋₄ alkyl radical,

with the provisos that

the compounds of formula (E2) contain only one bridging group Y per molecule and

the compounds of formula (E2) contain at least one amino group bearing at least one hydrogen atom.

According to the invention, the substituents used in formula (E2) are as defined in the foregoing.

Preferred binuclear primary intermediates corresponding to formula (E2) are, in particular, N,N′-bis-(β-hydroxyethyl)-N,N′-bis-(4′-aminophenyl)-1,3-diaminopropanol, N,N′-bis-(β-hydroxyethyl)-N,N′-bis-(4′-aminophenyl)-ethylenediamine, N,N′-bis-(4-aminophenyl)-tetramethylene diamine, N,N′-bis-(β-hydroxyethyl)-N,N′-bis-(4′-aminophenyl)-tetramethylene diamine, N,N′-bis-(4-methylaminophenyl)-tetramethylene diamine, N,N′-bis-(ethyl)-N,N′-bis-(4′-amino-3′-methylphenyl)-ethylenediamine, bis-(2-hydroxy-5-aminophenyl)-methane, 1,4-bis-(4′-aminophenyl)-diazacycloheptane, N,N′bis-(2-hydroxy-5-aminobenzyl)-piperazine, N-(4′-aminophenyl)-p-phenylenediamine and 1,10-bis-(2′,5′-diaminophenyl)-1,4,7,10-tetraoxadecane and physiologically compatible salts thereof.

Most particularly preferred binuclear primary intermediates corresponding to formula (E2) are N,N′-bis-(β-hydroxyethyl)-N,N′-bis-(4′-aminophenyl)-1,3-diaminopropan-2-ol, bis-(2-hydroxy-5-aminophenyl)-methane, N,N′-bis-(4′-aminophenyl)-1,4-diazacycloheptane and 1,10-bis-(2′,5′-diaminophenyl)-1,4,7,10-tetraoxadecane or a physiologically compatible salt thereof.

In another preferred embodiment of the invention, a p-aminophenol derivative or a physiologically compatible salt thereof is used as the primary intermediate. Particularly preferred p-aminophenol derivatives correspond to formula (E3):

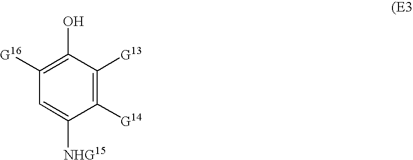

in which

G¹³ stands for a hydrogen atom, a halogen atom, a C₁₋₄ alkyl radical, a C₁₋₄ monohydroxyalkyl radical, a (C₁₋₄)-alkoxy-(C₁₋₄)-alkyl radical, a C₁₋₄ aminoalkyl radical, a hydroxy-(C₁₋₄)-alkylamino radical, a C₁₋₄ hydroxyalkoxy radical, a C₁₋₄ hydroxyalkyl-(C₁₋₄)-aminoalkyl radical or a (di-C₁₋₄-alkylamino)-(C₁₋₄)-alkyl radical,

G¹⁴ stands for a hydrogen atom or a halogen atom, a C₁₋₄ alkyl radical, a C₁₋₄ monohydroxyalkyl radical, a C₂₋₄ polyhydroxyalkyl radical, a (C₁₋₄)-alkoxy-(C₁₋₄)-alkyl radical, a C₁₋₄ aminoalkyl radical or a C₁₋₄ cyanoalkyl radical,

G¹⁵ stands for hydrogen, a C₁₋₄ alkyl radical, a C₁₋₄ monohydroxyalkyl radical, a C₂₋₄ polyhydroxyalkyl radical, a phenyl radical or a benzyl radical and

G¹⁶ stands for hydrogen or a halogen atom.

According to the invention, the substituents used in formula (E3) are defined as in the foregoing.

Preferred p-aminophenols corresponding to formula (E3) are, in particular, p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(2-hydroxyethoxy)-phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)-phenol, 4-amino-2-fluorophenol, 4-amino-2-chlorophenol, 2,6-dichloro-4-aminophenol, 4-amino-2-((diethylamino)methyl)phenol and physiologically compatible salts thereof.

Most particularly preferred compounds corresponding to formula (E3) are p-aminophenol, 4-amino-3-methylphenol, 4-amino-2-aminomethylphenol and 4-amino-2-((diethylamino)methyl)phenol.

In addition, the primary intermediate may be selected from o-aminophenol and its derivatives such as, for example, 2-amino-4-methylphenol or 2-amino-4-chlorophenol.

The primary intermediate may also be selected from heterocyclic primary intermediates such as, for example, pyridine, pyrimidine, pyrazole, pyrazole-pyrimidine derivatives and physiologically compatible salts thereof.

Preferred pyridine derivatives are, in particular, the compounds described in GB 1,026,978 and GB 1,153,196, such as 2,5-diaminopridine, 2-(4-methoxyphenyl)-amino-3-aminopyridine, 2,3-diamino-6-methoxypyridine, 2-(β-methoxyethyl)-amino-3-amino-6-methoxypyridine and 3,4-diaminopyridine.

Preferred pyrimidine derivatives are, in particular, the compounds described in DE 2359399, JP 02019576 A2 and WO 96/15765, such as 2,4,5,6-tetraaminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine and 2,5,6-triaminopyridine.

Preferred pyrazole derivatives are, in particular, the compounds described in patents DE 3843892 and DE 4133957 and in patent applications WO 94/08969, WO 94/08970, EP 740931 and DE 19543988, such as 4,5-diamino-1-methylpyrazole, 4,5-diamino-1-(β-hydroxyethyl)-pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methyl pyrazole, 4,5-diamino-3-tert.butyl-1-methylpyrazole, 4,5-diamino-1-tert.butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)-pyrazole, 4,5diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5diamino-3-methyl-1-isopropylpyrazole, 4-amino-5-(2′-aminoethyl)-amino-1,3-dimethylpyrazole, 3,4,5-triaminopyrazole, 1-methyl-3,4,5-triaminopyrazole, 3,5-diamino-1-methyl-4-methylaminopyrazole and 3,5diamino-4-(β-hydroxyethyl)-amino-1-methylpyrazole.

Preferred pyrazole-pyrimidine derivatives are, in particular, the derivatives of pyrazole-[1,5-a]-pyrimidine corresponding to formula (E4) below and tautomeric forms thereof where a tautomeric equilibrium exists:

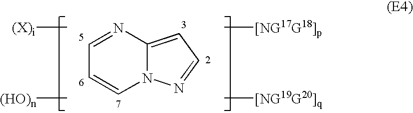

in which

G¹⁷, G¹⁸, G¹⁹ and G²⁰ independently of one another stand for a hydrogen atom, a C₁₋₄ alkyl radical, an aryl radical, a C₁₋₄ hydroxyalkyl radical, a C₂₋₄ polyhydroxyalkyl radical, a (C₁₋₄)-alkoxy-(C₁₄)-alkyl radical, a C₁₋₄ aminoalkyl radical which may optionally be protected by an acetylureide or sulfonyl radical, a (C₁₋₄)-alkylamino-(C₁₋₄)-alkyl radical, a di[(C₁₋₄)-alkyl]-(C₁₋₄)-aminoalkyl radical, the dialkyl radicals optionally forming a carbon cycle or a heterocycle with 5 or 6 links, a C₁₋₄ hydroxyalkyl or a di-(C₁₋₄)-[hydroxyalkyl]-(C₁₋₄)-aminoalkyl radical;

the X radicals independently of one another stand for a hydrogen atom, a C₁₋₄ alkyl radical, an aryl radical, a C₁₋₄ hydroxyalkyl radical, a C₂₋₄ polyhydroxyalkyl radical, a C₁₋₄ aminoalkyl radical, a (C₁₋₄)alkylamino-(C₁₋₄)-alkyl radical, a di[(C₁₋₄)-alkyl]-(C₁₋₄)-aminoalkyl radical, the dialkyl radicals optionally forming a carbon cycle or a heterocycle with 5 or 6 links, a C₁₋₄ hydroxyalkyl or a di-(C₁₋₄)-[hydroxyalkyl]-(C₁₋₄)-aminoalkyl radical, an amino radical, a C₁₋₄ alkyl or a di-(C₁₋₄ hydroxyalkyl)-amino radical, a halogen atom, a carboxylic acid group or a sulfonic acid group,

i has the value 0, 1, 2 or 3,

p has the value 0 or 1,

q has the value 0 or 1 and

n has the value 0 or 1,

with the proviso that

the sum of p+q is not 0,

where p+q=2, n has the value 0 and the groups NG¹⁷G¹⁸ and NG¹⁹G²⁰ occupy the (2,3); (5,6); (6,7); (3,5) or (3,7) positions;

where p+q=1, n has the value 1 and the groups NG¹⁷G¹⁸ (or NG¹⁹G²⁰) and the group OH occupy the (2,3); (5,6); (6,7); (3,5) or (3,7) positions.

The substituents used in formula (E4) are as defined in the foregoing.

If the pyrazole-[1,5-a]-pyrimidine corresponding to formula (V) above contains a hydroxy group in one of the positions 2, 5 or 7 of the ring system, a tautomeric equilibrium exists as illustrated, for example, in the following scheme:

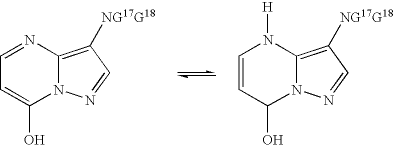

Among the pyrazole-[1,5-a]-pyrimidines corresponding to formula (V) above, the following may be particularly mentioned:

pyrazole-[1,5-a]-pyrimidine-3,7-diamine;

2,5-dimethylpyrazole-[1,5-a]-pyrimidine-3,7-diamine;

pyrazole-[1,5-a]-pyrimidine-3,5-diamine;

2,7-dimethylpyrazole-[1,5-a]-pyrimidine-3,5-diamine;

3-aminopyrazole-[1,5-a]-pyrimidin-7-ol;

3-aminopyrazole-[1,5-a]-pyrimidin-5-ol;

2-(3-aminopyrazole-[1,5-a]-pyrimidin-7-ylamino)-ethanol;

2-(7-aminopyrazole-[1,5-a]-pyrimidin-3-ylamino)-ethanol;

2-[(3-aminopyrazole-[1,5-a]-pyrimidin-7-yl)-(2-hydroxyethyl)-amino]-ethanol;

2-[(7-aminopyrazole-[1,5-a]-pyrimidin-3-yl)-(2-hydroxyethyl)-amino]-ethanol;

5,6-dimethylpyrazole-[1,5-a]-pyrimidine-3,7-diamine;

2,6-dimethylpyrazole-[1,5-a]-pyrimidine-3,7-diamine;

2,5,N7,N7-tetramethylpyrazole-[1,5-a]-pyrimidine-3,7-diamine;

and physiologically compatible salts thereof and tautomeric forms thereof where a tautomeric equilibrium exists.

The pyrazole-[1,5-a]-pyrimidines corresponding to formula (V) above may be prepared by cyclization from an aminopyrazole or from hydrazine, as described in the literature.

In addition, the preparations according to the invention may contain cationic dye precursors of the primary intermediate and/or secondary intermediate type as described, for example, in WO-A1-99/03 819, WO-A2-99/03 834, WO-A1-99/03 836, WO-A1-99/48 856, WO-A1-99/48 874, WO-A1-99/48 875, WO-A2-00/42 971, WO-A1-00/42 979, WO-A1-00/42 980, WO-A1-00/43 356, WO-A1-00/43 367, WO-A1-00/43 368, WO-A1-00/43 386, WO-A1-00/43 388, WO-A1-00/43 389, WO-A1-00/43 396, EP-A1-0 984 006, EP-A1-0 984 007 and EP-A1-0 989 128.

Particularly preferred cationic dye precursors are

[2-(2′,5′-diaminophenoxy)-ethyl]-diethylmethyl ammonium chloride,

[2-(4′-aminophenylamino)-propyl]-trimethyl ammonium chloride,

[4-(4′-aminophenylamino)-pentyl]-diethyl-(2-hydroxyethyl)-ammonium chloride,

1-{[5′-amino-2′-(2″-hydroxyethylamino)-phenylcarbamoyl]-methyl}-1,4-dimethyl piperazin-1-ium chloride,

1,4-bis-l-{3-[3′-(2″,5″-diaminophenoxy)-propyl]-3H-imidazol-1-ium}-butane dichloride,

1,3-bis-[3′-(2″,5″-diaminophenoxy)-propyl]-3H-imidazol-1-ium chloride,

N,N′-bis-[3-N-methyl-4-N-(4′-aminoaniline)-ethyl]-1,1,4,4-tetramethyl diammonium 1,3-propane dibromide,

1,3-bis-1-{3-{3′-[(4″-amino-3″-methylaniline)-N-propyl]}-3H-imidazol-1-ium}-propane dichloride,

1,3-bis-1-{3-{3′-[(4″-aminoaniline)-N-propyl]}-3H-imidazol-1-ium}-propane dichloride,

1,3-bis-1-{3-{3′-[(4″-amino-2″-methylaniline)-N-propyl]}-3H-imidazol-1-ium}-propane dichloride,

3-[3-(4′-aminophenylamino)-propyl]-l-methyl-3H-imidazol-1-ium chloride,

[3-(2′,5′-diaminophenoxy)-propyl]-3-methyl-3H-imidazol-1-ium chloride,

3-[3-(4′-amino-3′-methylphenylamino)-propyl]-l-methyl-3H-imidazol-1-ium chloride,

3-[3-(4′-amino-2′-methylphenylamino)-propyl]-l-methyl-3H-imidazol-1-ium chloride,

1-[2-(4′-amino-2′-methoxyphenylamino)-ethyl]-3-methyl-3H-imidazol-1-ium chloride,

3-[3-(4′-amino-2′-fluorophenylamino)-propyl]-l-methyl-3H-imidazol-1-ium chloride,

3-[3-(4′-amino-2′-cyanophenylamino)-propyl]-1-methyl-3H-imidazol-1-ium chloride,

3-[2-(2′,5′-diaminophenyl)-ethyl]-l-methyl-3H-imidazol-1-ium chloride,

1-{2-[(4′-aminophenyl)-ethylamino]-ethyl}-3-methyl-3H-imidazol-1-ium chloride,

N,N-bis-[2-(3′-methyl-3H-imidazol-1-ium)-ethyl]-4-aminoaniline chloride,

3-[2-(4′-aminophenylamino)-butyl]-l-methyl-3H-imidazol-1-ium chloride,

[2-(2′,4′-diaminophenoxy)-ethyl]-diethylmethyl ammonium chloride,

1-[3-(2′,4′-diaminophenoxy)-propyl]-3-methyl-3H-imidazol-1-ium chloride,

1-[(3′-hydroxy-4′-methylphenylcarbamoyl)-methyl]-3-methyl-3H-imidazol-1ium chloride,

1,4-bis-1-{3-[3-(2′,4′-diaminophenoxy)-propyl]-3H-imidazol-1-ium}-butane dichloride,

3-[(3′-hydroxy-4′-methansulfonylaminophenylcarbamoyl)-methyl]-1-methyl3H-imidazol-1-ium chloride,

3-[(3′,5′-dichloro-2′-hydroxy-4′-methylphenylcarbamoyl)-methyl]-1-methyl-3H-imidazol-1-ium chloride,

1-[(3′,5′-dichloro-2′-hydroxy-4′-methylphenylcarbamoyl)-methyl]-1,4-dimethyl piperazin-1-ium chloride,

3-[(4′-acetylamino-2′-hydroxyphenylcarbamoyl)-methyl]-1-methyl-3H-imidazol-1-ium chloride,

4-{3-[(3′-hydroxynaphthalene-2′-carbonyl)-amino]-propyl}-4-methyl-morpholin-4-ium iodide,

3-[(1′-hydroxynaphthalen-2′-ylcarbamoyl)-methyl]-1-methyl-3H-imidazol-1 ium chloride,

3-[(5′-acetylamino-1′-hydroxynaphthalen-2′-ylcarbamoyl)-methyl]-1-methyl-3H-imidazol-1-ium chloride,

3-[(1′-hydroxy-5′-methanesulfonylaminonaphthalen-2′-ylcarbamoyl)-methyl]-1-methyl-3H-imidazol-1-ium chloride,

[3-(4′-amino-2′,5′-dimethyl-2H-pyrazol-3′-ylamino)-propyl]-(2-hydroxyethyl)-dimethylammonium chloride,

1,3-bis-[(2′-hydroxy-4′-methylphenylcarbamoyl)-methyl]-3H-imidazol-1-ium chloride,

1-[2-(6′-aminobenzo[1,3]dioxol-5′-ylamino)-ethyl]-3-methyl-3H-imidazol-1-ium chloride,

3-[2-(6′-aminobenzo[1,3]dioxol-5′-ylamino)-ethyl]-1-(4-{3-[2-(6″-aminobenzo[1,3]dioxol-5′-ylamino)-ethyl]-3H-imidazol-1-ium}-butyl)-3H-imidazol-1-ium dichloride,

3-[3-(3′-amino-5′-methylpyrazolo[1,5-a]pyrimidin-7′-ylamino)-propyl]-1-(2hydroxyethyl)-3-H-imidazol-1-ium chloride,

1,3-bis-1-{3-{3-[(2′-aminoaaniline)-N-propyl]}-3H-imidazol-1-ium}-propane dibromide,

N,N′-bis-[3-N-(2′-aminoaniline)-N-propyl]-1,1,3,3-tetramethyldiammonium 1,3-propane dibromide,

3-[3-(2′-aminophenylamino)-propyl]-1-methyl-3H-imidazol-1-ium chloride,

[2-(2′-aminophenylamino)-ethyl]-trimethylammonium chloride and

3-(4′-hydroxy-1′-methyl-1H-indol-5′-ylmethyl)-1-methyl-pyridinium methosulfate.

According to the invention, other preferred colorants contain at least one secondary intermediate. m-Phenylenediamine derivatives, naphthols, resorcinol and resorcinol derivatives, pyrazolones and m-aminophenol derivatives are generally used as secondary intermediates. Particularly suitable secondary intermediates are 1-naphthol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis-(2,4diaminophenoxy)-propane, 2-chlororesorcinol, 4-chlororesorcinol, 2-chloro-6-methyl-3-aminophenol, 2-amino-3-hydroxypyridine, 2-methyl resorcinol, 5-methyl resorcinol and 2-methyl-4-chloro-5-aminophenol.

According to the invention, preferred secondary intermediates are

m-aminophenol and derivatives thereof such as, for example, 5amino-2-methylphenol, 3-amino-2-chloro-6-methylphenol, 2hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′hydroxyethyl)-amino-2-methylphenol, 3-(diethylamino)-phenol, N-cyclopentyl-3-aminophenol, 1,3-dihydroxy-5-(methylamino)-benzene, 3-(ethylamino)-4-methylphenol and 2,4-dichloro-3aminophenol,

o-aminophenol and derivatives thereof,

m-diaminobenzene and derivatives thereof such as, for example, 2,4-diaminophenoxyethanol, 1,3-bis-(2,4-diaminophenoxy)-propane, 1-methoxy-2-amino-4-(2′-hydroxyethylamino)-benzene, 1,3-bis-(2,4diaminophenyl)-propane, 2,6-bis-(2-hydroxyethylamino)-1-methylbenzene and 1-amino-3-bis-(2′-hydroxyethyl)-aminobenzene,

o-diaminobenzene and derivatives thereof such as, for example, 3,4diaminobenzoic acid and 2,3-diamino-1-methylbenzene,

di- and trihydroxybenzene derivatives such as, for example, resorcinol, resorcinol monomethyl ether, 2-methyl resorcinol, 5-methyl resorcinol, 2,5-dimethyl resorcinol, 2-chlororesorcinol, 4chlororesorcinol, pyrogallol and 1,2,4-trihydroxybenzene,

pyridine derivatives such as, for example, 2,6-dihydroxypyridine, 2-amino-3-hydroxypyridine, 2-amino-5-chloro-3-hydroxypyridine, 3amino-2-methylamino-6-methoxypyridine, 2,6-dihydroxy-3,4dimethylpyridine, 2,6-dihydroxy-4-methylpyridine, 2,6-diaminopyridine, 2,3-diamino-6-methoxypyridine and 3,5-diamino-2,6-dimethoxypyridine,

naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihdroxynaphthalene, 1,7-dihdroxynaphthalene, 1,8-dihdroxynaphthalene, 2,7-dihdroxynaphthalene and 2,3-dihdroxynaphthalene,

morpholine derivatives such as, for example, 6-hydroxybenzomorpholine and 6-aminobenzomorpholine,

quinoxaline derivatives such as, for example, 6-methyl-1,2,3,4-tetrahydroquinoxaline,

pyrazole derivatives such as, for example, 1-phenyl-3-methylpyrazol-5-one,

indole derivatives such as, for example, 4-hydroxyindole, 6-hydroxyindole and 7-hydroxyindole,

pyrimidine derivatives such as, for example, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4hydroxy-6-methylpyrimidine and 4,6-dihydroxy-2-methylpyrimidine or

methylenedioxybenzene derivatives such as, for example, 1-hydroxy-3,4-methylenedioxybenzene, 1-amino-3,4-methylenedioxybenzene and 1-(2′-hydroxyethyl)-amino-3,4-methylenedioxybenzene.

Particularly preferred secondary intermediates are 1-naphthol, 1,5-, 2,7- and 1,7-dihydroxynaphthalene, 3-aminophenol, 5-amino-2methylphenol, 2-amino-3-hydroxypyridine, resorcinol, 4-chlororesorcinol, 2-chloro-6-methyl-3-aminophenol, 2-methyl resorcinol, 5-methyl resorcinol, 2,5-dimethyl resorcinol and 2,6-dihydroxy-3,4-dimethylpyridine.

The oxidation dye precursors or the substantive dyes do not have to be single compounds. On the contrary, other components may be present in small quantities in the hair colorants according to the invention due to the processes used to produce the individual dyes providing these other components do not adversely affect the coloring result or have to be ruled out for other reasons, for example toxicological reasons.

So far as the dyes suitable for use in the hair colorants and tinting preparations according to the invention are concerned, reference is also expressly made to the work by Ch. Zviak, The Science of Hair Care, Chapter 7 (pages 248-250; substantive dyes) and Chapter 8, pages 264-267; oxidation dye precursors), published as Volume 7 of the Series “Dermatology” (Ed.: Ch. Culnan and H. Maibach), Marcel Dekker Inc., New York/Basle, 1986, and to the “Europäisch Inv ntar d r Kosmetik-Rohstoffe” published by the Europäische Gemeinschaft and available in disk form from the Bundesverband Deutscher Industrie- und Handelsunternehmen für Arzneimittel, Reformwaren und Körperpflegemittel d.V., Mannheim.

If the dye precursors are amino compounds, the known acid addition salts may be prepared from them in the usual way. Accordingly, all statements in this specification and hence the claimed scope of protection relate both to the compounds present in free from and to their water-soluble physiologically compatible salts. Examples of such salts are the hydrochlorides, the hydrobromides, the sulfates, the phosphates, the acetates, the propionates, the citrates and the lactates.

The oxidation dye precursors are present in the preparations according to the invention in quantities of preferably 0.01 to 20% by weight and more particularly 0.5 to 5% by weight, based on the preparation as a whole.

Preferred precursors of “nature-analogous” dyes are indoles and indolines which contain at least one hydroxy or amino group, preferably as a substituent on the six ring. These groups may carry further substituents, for example in the form of an etherification or esterification of the hydroxy group or an alkylation of the amino group. In a second preferred embodiment, the colorants contain at least one indole and/or indoline derivative.

Particularly suitable precursors of “nature-analogous” hair dyes are derivatives of 5,6-dihydroxyindoline corresponding to formula (Ia):

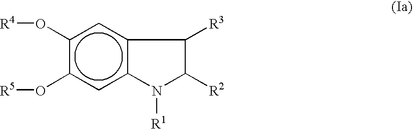

in which—independently of one another—

R¹ is hydrogen, a C₁₋₄ alkyl group or a C₁₋₄ hydroxyalkyl group,

R² is hydrogen or a —COOH group, the —COOH group optionally being present as a salt with a physiologically compatible cation,

R³ is hydrogen or a C₁₋₄ alkyl group,

R⁴ is hydrogen, a C₁₋₄ alkyl group or a group —CO—R⁶, where R⁶ is a C₁₋₄ alkyl group, and

R⁵ is one of the groups mentioned for R⁴,

and physiologically compatible salts of these compounds with an organic or inorganic acid.

Particularly preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid and 6-hydroxyindoline, 6-aminoindoline and 4aminoindoline.

Within this group, particular emphasis is placed on N-methyl-5,6dihydroxyindoline, N-ethyl-5,6-dihydroxyindoline, N-propyl-5,6-dihydroxyindoline, N-butyl-5,6-dihydroxyindoline and, in particular, 5,6-dihydroxyindoline.

Other particularly suitable precursors of “nature-analogous” hair dyes are derivatives of 5,6-dihydroxyindole corresponding to formula (Ib):

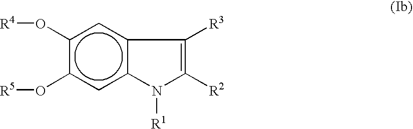

in which—independently of one another—

R¹ is hydrogen, a C₁₋₄ alkyl group or a C₁₋₄ hydroxyalkyl group,

R² is hydrogen or a —COOH group, the —COOH group optionally being present as a salt with a physiologically compatible cation,

R³ is hydrogen or a C₁₋₄ alkyl group,

R⁴ is hydrogen, a C₁₋₄ alkyl group or a group —CO—R⁶, where R⁶ is a C₁₋₄ alkyl group, and

R⁵ is one of the groups mentioned for R⁴,

and physiologically compatible salts of these compounds with an organic or inorganic acid.

Particularly preferred derivatives of indole are 5,6-dihydroxyindole, N-methyl-5,6-dihydroxyindole, N-ethyl-5,6-dihydroxyindole, N-propyl-5,6dihydroxyindole, N-butyl-5,6-dihydroxyindole, 5,6-dihydroxyindole-2carboxylic acid, 6-hydroxyindole, 6-aminoindole and 4-aminoindole.

Within this group, particular emphasis is placed on N-methyl-5,6dihydroxyindole, N-ethyl-5,6-dihydroxyindole, N-propyl-5,6-dihydroxyindole, N-butyl-5,6-dihydroxyindole and, in particular, 5,6-dihydroxyindole.

The indoline and indole derivatives may be used both as free bases and in the form of their physiologically compatible salts with inorganic or organic acids, for example hydrochlorides, sulfates and hydrobromides, in the colorants used in the process according to the invention. The indole or indoline derivatives are present in these colorants in quantities of normally 0.05 to 10% by weight and preferably 0.2 to 5% by weight.

In another preferred embodiment of the invention, the indoline or indole derivative may be used in combination with at least one amino acids or an oligopeptide in hair colorants. The amino acid is advantageously an α-amino acid. Most particularly preferred α-amino acids are arginine, ornithine, lysine, serine and histidine, especially arginine.

Besides the dye precursors, the preparations according to the invention may additionally contain at least one substantive dye for the purpose of obtaining the required color effects. Substantive dyes are typically nitrophenylenediamines, nitroaminophenols, azo dyes, anthraquinones or indophenols. Preferred substantive dyes are the compounds known under the International names or commercial names of HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 12, Disperse Blue 3, HC Violet 1, Disperse Violet 1, Disperse Violet 4, Acid Violet 43, Disperse Black 9 and Acid Black 52 and also 1,4-diamino-2-nitrobenzene, 2-amino-4-nitrophenol, 1,4-bis-(β-hydroxyethyl)-amino-2-nitrobenzene, 3-nitro-4-(βhydroxyethyl)-aminophenol, 2-(2′-hydroxyethyl)-amino-4,6-dinitrophenol, 1-(2′-hydroxyethyl)-amino-4-methyl-2-nitrobenzene, 1-amino-4-(2′hydroxyethyl)-amino-5-chloro-2-nitrobenzene, 4-amino-3-nitrophenol, 1-(2′-ureidoethyl)-amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, hydroxyethyl-2-nitrotoluidine, picramic acid and salts thereof, 2-amino-6-chloro-4-nitrophenol, 4-ethylamino-3-nitrobenzoic acid and 2-chloro-6-ethylamino-1-hydroxy-4-nitrobenzene.

In addition, the preparations according to the invention may contain a cationic substantive dye. Particularly preferred are

(i) cationic triphenylmethane dyes such as, for example, Basic Blue 7, Basic Blue 26, Basic Violet 2 and Basic Violet 14,

(ii) aromatic systems substituted by a quaternary nitrogen group such as, for example, Basic Yellow 57, Basic Red 76, Basic Blue 99, Basic Brown 16 and Basic Brown 17 and

(iii) substantive dyes containing a heterocycle with at least one quaternary nitrogen atom as disclosed, for example, in EP-A2 998 908, to which reference is specifically made at this juncture, in claims 6 to 11.

Preferred cationic substantive dyes of group (iii) are, in particular, the following compounds:

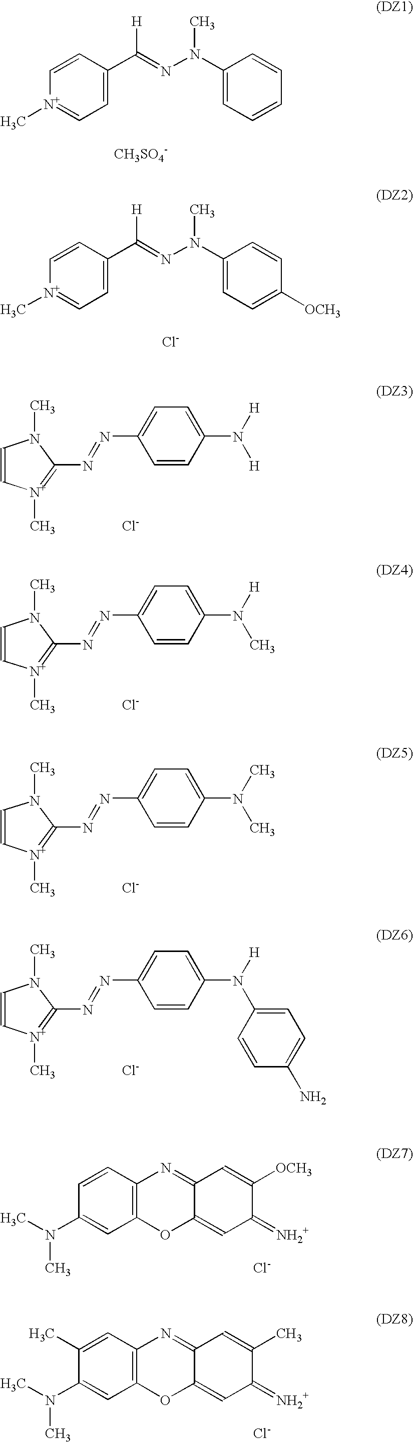

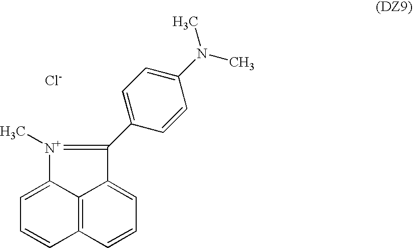

The compounds corresponding to formula (DZ1), (DZ3) and (DZ5) are most particularly preferred cationic substantive dyes of group (iii).

The preparations according to the invention of this embodiment contain the substantive dyes in a quantity of preferably 0.01 to 20% by weight, based on the colorant as a whole.

The preparations according to the invention may also contain naturally occurring dyes such as, for example, henna red, henna neutral, henna black, camomile blossom, sandalwood, black tea, black alder bark, sage, logwood, madder root, catechu, sedre and alkanet.

Enzymes

In addition, the preparations according to the invention contain at least one enzyme capable of catalyzing the oxidation of the dye precursors as a second compulsory component. Although, in principle, any enzymes capable of catalyzing this process are suitable for the purposes of the invention, enzymes which have proved to be particularly suitable

(i) produce small quantities of hydrogen peroxide in situ,

(ii) directly oxidize the dye precursors with the aid of atmospheric oxygen or

(iii) accelerate the oxidation of the dye precursors by hydrogen peroxide.

The behavior described in (i) is typically exhibited by oxidases which react with their particular substrate to form hydrogen peroxide. Examples of such enzymes are glucose oxidase (EC No. 1.1.3.4), alcohol oxidase. (EC No. 1.1.3.13), oxidase for secondary alcohols (EC No. 1.1.3.18), oxidase for long-chain alcohols (EC No. 1.1.3.20), glycerol-3-phosphate oxidase (EC No. 1.1.3.21), glycolate oxidase (EC No. 1.1.3.15), methanol oxidase (EC No. 1.1.3.31), vanillyl alcohol oxidase (EC No. 1.1.3.38), pyruvate oxidase (EC No. 1.1.2.3), oxalate oxidase (EC No. 1.2.3.4), cholesterol oxidase (EC No. 1.1.3.6), uricase (EC No. 1.7.3.3), lactate oxidase (EC No. 1.13.12.4), xanthine oxidase (EC No. 1.1.3.22), pyranose oxidase (EC No. 1.1.3.10), aminoacid oxidases (EC No. 1.4.3.2, EC No. 1.4.3.3), acyl-CoA-oxidase (EC No. 1.3.3.6), glutamate oxidases (EC No. 1.4.3.7, EC No. 1.4.3.11), protein lysine-6-oxidase (EC No. 1.4.3.14), lysine oxidase (EC No. 1.4.3.14), sulfite oxidase (EC No. 1.8.3.1), catechol oxidase (EC No. 1.10.3.1), L-ascorbate oxidase (EC No. 1.10.3.3), choline oxidase (EC No. 1.1.3.17), monoamine oxidase (EC No. 1.4.3.4), diamine oxidase (EC No. 1.4.3.6), sarcosine oxidase (EC No. 1.5.3.1) and galactose oxidase (EC No. 1.1.3.9). Particularly preferred examples of these oxidases are uricase, glucose oxidase and choline oxidase. Uricase is a most particularly preferred class (i) enzyme. For these enzymes to be able to catalyze the coloring process according to the invention, the preparations according to the invention must always contain the corresponding substrates in a sufficient quantity.

Enzymes which directly oxidize the dye precursors with the aid of atmospheric oxygen, (ii), are, for example, the laccases (EC No. 1.10.3.2), the tyrosinases (EC No. 1.10.3.1), ascorbate oxidase (EC No. 1.10.3.3), the bilirubin oxidases (EC No. 1.3.3.5) and phenoloxidases of the acremonia, stachybotrys or pleurotus type. According to the invention, the laccases are most particularly preferred class (ii) enzymes.

The peroxidases (EC No. 1.11.1.7) come into category (iii) of preferred enzymes according to the invention. Peroxidases allow coloring even with minimal quantities of hydrogen peroxide. It does not matter whether small quantities of hydrogen peroxide are incorporated in the formulation or whether the hydrogen peroxide is formed in situ by the enzymes listed under (i). Peroxidase which can be obtained from horseradish is particularly preferred for the purposes of the invention.

The enzymes may be incorporated in the colorant itself. However, they are preferably made up separately from the dye precursors and are mixed with the actual colorant just before application. If the enzymes are to be incorporated in the colorant as a one-component system, it is preferred in accordance with the invention to make up the system in the absence of oxygen.

In another preferred embodiment of the invention, the colorant can be made up without propellents, i.e. not as a foam formulation.

The enzyme is preferably used in a quantity of 0.0001 to 1% by weight, based on the amount of protein in the enzyme and the colorant as a whole.

Sugar Surfactants

The preparations according to the invention contain at least one sugar surfactant as a third compulsory component (C).

In a first embodiment (C1), the sugar surfactant is an alkyl or alkenyl oligoglycoside. These sugar surfactants are known nonionic surfactants corresponding to formula (II):

R¹O—[G]ₚ (II)

where R¹ is an alkyl or alkenyl group containing 4 to 22 carbon atoms, G is a sugar unit containing 5 or 6 carbon atoms and p is a number of 1 to 10. They may be obtained by the relevant methods of preparative organic chemistry. The overviews presented by Bierman et al. in Starch/Stärke 45, 281 (1993), by B. Salka in Cosm. Toil. 108, 89 (1993) and by J. Kahre in SÖFW-Journal No. 8, 598 (1995) are cited as representative of the extensive literature available on this subject.

The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in general formula (II) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view.

The alkyl or alkenyl radical R¹ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen′s oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₂ alcohol as an impurity, and also alkyl oligoglucosides based on technical C_{9/11} oxoalcohols (DP=1 to 3) are preferred. In addition, the alkyl or alkenyl radical R¹ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} coconut oil fatty alcohol having a DP of 1 to 3 are preferred.

In a second embodiment (C2) of the invention, the sugar surfactant is a fatty acid-N-alkyl polyhydroxyalkylamide, a nonionic surfactant corresponding to formula (III):

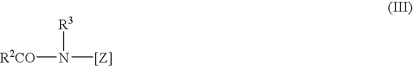

in which R²CO is an aliphatic acyl group containing 6 to 22 carbon atoms, R³ is hydrogen, an alkyl or hydroxyalkyl group containing 1 to 4 carbon atoms and [Z] is a linear or branched polyhydroxyalkyl group containing 3 to 12 carbon atoms and 3 to 10 hydroxyl groups. The fatty acid-N-alkyl polyhydroxyalkylamides are known compounds which may normally be obtained by reductive amination of a reducing sugar with ammonia, an alkylamine or an alkanolamine and subsequent acylation with a fatty acid, a fatty acid alkyl ester or a fatty acid chloride. Processes for their production are described in U.S. Pat. No. 1,985,424, in U.S. Pat. No. 2,016,962 and in U.S. Pat. No. 2,703,798 and in International patent application WO 92/06984. An overview of this subject by H. Kelkenberg can be found in Tens. Surf. Det. 25, 8 (1988). The fatty acid-N-alkyl polyhydroxyalkylamides are preferably derived from reducing sugars containing 5 or 6 carbon atoms, more particularly from glucose. Accordingly, the preferred fatty acid-N-alkyl polyhydroxyalkylamides are fatty acid-N-alkyl glucamides which correspond to formula (IV):

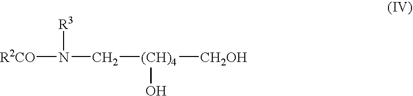

Preferred fatty acid-N-alkyl polyhydroxyalkylamides are glucamides corresponding to formula (IV) in which R³ is hydrogen or an alkyl group and R²CO represents the acyl component of caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid or erucic acid or technical mixtures thereof. Fatty acid-N-alkyl glucamides (IV) obtained by reductive amination of glucose with methylamine and subsequent acylation with lauric acid or C_{12/14} coconut oil fatty acid or a corresponding derivative are particularly preferred. In addition, the polyhydroxyalkylamides may also be derived from maltose and palatinose.

The sugar surfactant is present in the preparations used in accordance with the invention in quantities of preferably 0.1 to 20% by weight and more particularly 0.5 to 5% by weight, based on the preparation as a whole.

Reducing Agents

In addition, the preparations according to the invention preferably contain a reducing agent. Examples of preferred reducing agents for the purposes of the invention are sodium sulfite, ascorbic acid, thioglycolic acid and derivatives thereof, sodium thionite, alkali metal citrate salts and N-acetyl-L-cysteine. Most particularly preferred reducing agents are alkali metal citrate salts, more particularly sodium citrate, and N-acetyl-L-cysteine. N-acetyl-L-cysteine is a most particularly preferred alkalizing agent.

Fatty Acid Partial Glyceride

The preparations according to the invention may contain fatty acid partial glycerides as another component. These fatty acid partial glycerides are monoglycerides, diglycerides and technical mixtures thereof. Where technical products are used, small quantities of triglycerides may also be present from the production process. The partial glycerides preferably correspond to formula (V):

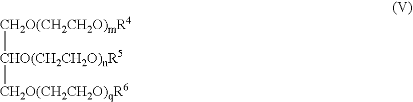

in which R⁴, R⁵ and R⁶ independently of one another represent hydrogen or a linear or branched, saturated and/or unsaturated acyl group containing 6 to 22, preferably 12 to 18, carbon atoms, with the proviso that at least one of these substituents is an acyl group and at least one is hydrogen. The sum (m+n+q) is 0 or a number of 1 to 100 and preferably 0 or a number of 5 to 25. Preferably, R⁴ is an acyl group and R⁵ and R⁶ are hydrogen atoms and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. Oleic acid monoglycerides are preferably used.

The fatty acid partial glyceride is present in the preparations used in accordance with the invention in quantities of preferably 0.1 to 20% by weight and more particularly 0.5 to 2% by weight, based on the preparation as a whole.

The teaching according to the invention also encompasses embodiments in which the preparations-according to the invention contain several sugar surfactants and/or several fatty acid partial glycerides.

Thickeners

In addition, the preparations according to the invention may contain at least one thickener as a further, optional component. Basically, the choice of thickeners is unlimited. Although, in principle, both organic and purely inorganic thickeners may be used, the organic thickeners are preferred for the purposes of the invention.

In a first preferred embodiment, the thickener is an anionic synthetic polymer. Preferred anionic groups are the carboxylate group and the sulfonate group.

Examples of anionic monomers of which the polymeric anionic thickeners may consist are acrylic acid, methacrylic acid, crotonic acid, itaconic acid, maleic anhydride and 2-acrylamido-2-methylpropanesulfonic acid. The acidic groups may be completely or partly present as the sodium, potassium, ammonium, mono- or triethanolammonium salt. Preferred monomers are maleic anhydride and, in particular, 2-acrylamido2-methylpropanesulfonic acid and acrylic acid.

Preferred anionic homopolymers are uncrosslinked and crosslinked polyacrylic acids. Allyl ethers of pentaerythritol, of sucrose and of propylene can be preferred crosslinking agents. Such compounds are commercially available, for example, under the registered name of Carbopol®. Equally preferred is the homopolymer of 2-acrylamido-2-methylpropanesulfonic acid which is commercially available, for example, under the name of Rheothik® 11-80.

A preferred variant of this first embodiment is characterized by the use of copolymers of at least one anionic monomer and at least one nonionic monomer. The anionic monomers may be selected from the substances mentioned above. Preferred nonionic monomers are acrylamide, methacrylamide, acrylates, methacrylates, itaconic acid mono- and diesters, vinyl pyrrolidone, vinyl ethers and vinyl esters.

Preferred anionic copolymers are, for example, copolymers of acrylic acid, methacrylic acid or C₁₋₆ alkyl esters thereof which are marketed under the INCI Declaration Acrylates Copolymer. A preferred commercial product is, for example, Aculyn® 33 from Rohm & Haas. However, copolymers of acrylic acid, methacrylic or C₁₋₆ alkyl esters thereof and the esters of an ethylenically unsaturated acid and an alkoxylated fatty alcohol are also preferred. Suitable ethylenically unsaturated acids are, in particular, acrylic acid, methacrylic acid and itaconic acid. Suitable alkoxylated fatty alcohols are, in particular, Steareth-20 or Ceteth-20. Copolymers such as these are marketed by Rohm & Haas under the name of Aculyn® 22 and by National Starch under the names of Structure® 2001 and Structure® 3001.

Other preferred anionic copolymers are acrylic acid/acrylamide copolymers and, in particular, polyacrylamide copolymers with monomers containing sulfonic acid groups. A particularly preferred anionic copolymer consists of 70 to 55 mol-% acrylamide and 30 to 45 mol-% 2-acrylamido-2methylpropanesulfonic acid, the sulfonic acid group being completely or partly present as a sodium, potassium, ammonium, mono- or triethanolammonium salt. This copolymer may also be present in crosslinked form, polyolefinically unsaturated compounds, such as tetraallyloxythane, allyl sucrose, allyl pentaerythritol and methylene bisacrylamide preferably being used as crosslinking agents. One such polymer is present in the commercial products Sepigel® 305 and Simulgel® 600 from SEPPIC. The use of these compounds which, besides the polymer component, contain a hydrocarbon mixture (C₁₃₋₁₄ isoparaffin or isohexadecane) and a nonionic emulsifier (Laureth-7 or Polysorbate-80) has proved to be particularly advantageous for the purposes of the teaching according to the invention.

Other preferred thickeners are polymers of maleic anhydride and methylvinylether, particularly those with crosslinkages. A maleic acid/methylvinylether copolymer crosslinked with 1,9-decadiene is commercially obtainable under the name of Stabileze® QM.

In a second embodiment, the thickener is a cationic synthetic polymer. Preferred cationic groups are quaternary ammonium groups. Polymers where the quaternary ammonium group is attached by a C₁₋₄ hydrocarbon group to a polymer main chain made up of acrylic acid, methacrylic acid or derivatives thereof have proved to be particularly suitable.

Particularly preferred cationic polymeric thickeners are homopolymers corresponding to general formula (VI):

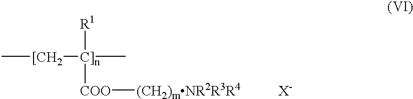

in which R¹=—H or —CH₃, R², R³ and R⁴ independently of one another are selected from C₁₋₄ alkyl, alkenyl or hydroxyalkyl groups, m=1, 2, 3 or 4, n is a natural number and X⁻ is a physiologically compatible organic or inorganic anion, and copolymers consisting essentially of the monomer units shown in formula (I) and nonionic monomer units. Among these polymers, those to which at least one of the following conditions applies:

R¹ is a methyl group,

R², R³ and R⁴ are methyl groups,

m has the value 2,

are particularly preferred for the purposes of the invention. The physiologically compatible counterion X⁻may be selected, for example, from halide ions, sulfate ions, phosphate ions, methosulfate ions and organic ions, such as lactate, citrate, tartrate and acetate ions. Halide ions, especially chloride, are preferred.

A particularly suitable homopolymer is the optionally crosslinked poly(methacryloyloxyethyltrimethylammoniumchloride) with the INCI name Polyquaternium-37. Crosslinking may be carried out, if desired, with polyolefinically unsaturated compounds, for example divinyl benzene, tetraallyl oxyethane, methylene bisacrylamide, diallyl ether, polyallyl polyglyceryl ether or allyl ethers of sugars or sugar derivatives, such as erythritol, pentaerythritol, arabitol, mannitol, sorbitol, sucrose or glucose. Methylene bisacrylamide is a preferred crosslinking agent.

The homopolymer is preferably used in the form of a nonaqueous polymer dispersion which should have a percentage polymer content of not less than 30% by weight. Such polymer dispersions are commercially available under the names of Salcare® SC 95 (ca. 50% polymer content, other component: mineral oil (INCI name: Mineral Oil) and tridecyl polyoxypropylene/polyoxyethylene ether (INCI name: PPG-1-Trideceth-6)) and Salcare® SC 96 (ca. 50% polymer content, other components: mixture of diesters of propylene glycol with a mixture of caprylic and capric acid (INCI name: Propylene Glycol Dicaprylate/Dicaprate) and tridecyl polyoxypropylene/polyoxyethylene ether (INCI name: PPG-1-Trideceth-6).

Nonionic thickeners of the polymeric fatty acid amide type disclosed, for example, in EP-A1-0 632 083 of EP-A2-0 959 066 are also suitable for the purposes of the invention.

Copolymers containing monomer units corresponding to formula (VI) preferably contain acrylamide, methacrylamide, C₁₋₄ alkyl acrylate and C₁₋₄ alkyl methacrylate as nonionic monomer units. Of these nonionic monomers, acrylamide is particularly preferred. As in the case of the homopolymers described above, these copolymers may also be crosslinked. According to the invention, a preferred copolymer is the crosslinked acrylamide/methacryloyloxyethyl trimethyl ammonium chloride copolymer. Copolymers in which the monomers are present in a ratio by weight of about 20:80 are commercially available as a ca. 50% nonaqueous polymer dispersion with the name of Salcare® SC 92.

A third preferred embodiment is characterized by the use of naturally occurring thickeners. Preferred thickeners of this embodiment are, for example, nonionic guar gums. According to the invention, both modified and unmodified guar gums may be used. Unmodified guar gums are marketed, for example, by Rhône-Poulenc under the name of Jaguar® C. According to the invention, preferred modified guar gums contain C₁₋₆ hydroxyalkyl groups. The hydroxymethyl, hydroxyethyl, hydroxypropyl and hydroxybutyl groups are preferred. Correspondingly modified guar gums are known and may be produced, for example, by reaction of guar gum with alkylene oxides. The degree of hydroxyalkylation which corresponds to the number of used alkylene oxide molecules in relation to the number of free hydroxy groups of the guar gum is preferably between 0.4 and 1.2. Correspondingly modified guar gums are commercially available from Rhône Poulenc under the names of Jaguar® HP 8, Jaguar® HP 60, Jaguar® HP 120, Jaguar® DC 293 and Jaguar® HP 105.

Other suitable natural thickeners are also known from the prior art. Accordingly, reference is expressly made to the book by Robert L. Davidson entitled “Handbook of Water Soluble Gums and Resins”, McGraw Hill Book Company (1980).

Other preferred gums of this embodiment are biosaccharide gums of microbial origin, such as the scleroglucan gums or xanthan gums, gums of vegetable exudates such as, for example, gum arabic, ghatti gum, karaya gum, tragacanth gum, carrageen gum, agar agar, locust bean gum, pectins, alginates, starch fractions and derivatives, such as amylose, amylopectin and dextrins.

However, nonionic fully synthetic polymers such as, for example, polyvinyl alcohol or polyvinyl pyrrolidone may also be used as thickeners according to the invention. Preferred nonionic fully synthetic polymers are marketed, for example, by BASF under the name of Luviskol®.

Anionic and nonionic thickening polymers are preferred for the purposes of the invention.

In a most particularly preferred embodiment of the present invention, cellulose and/or cellulose derivatives, such as methyl cellulose, carboxyalkyl celluloses and hydroxyalkyl celluloses for example, are used as thickeners.

Preferred hydroxyalkyl celluloses are, in particular, the hydroxyethyl celluloses marketed under the names of Cellosize® by Amerchol, Natrosol® by Hercules or Culminal® and Benecel® by Aqualon. The hydroxypropyl celluloses marketed, for example, under the name of Klucel® by Hercules are also suitable for the purposes of the invention. Suitable carboxyalkyl celluloses are, in particular, the carboxymethyl celluloses marketed under the names of Blanose® by Aqualon, Aquasorb® and Ambergum® by Hercules and Cellgon® by Montello.

Care Polymers

In another preferred embodiment, the preparations according to the invention additionally contain at least one care polymer. Care polymers are understood to be both natural and synthetic polymers which may be anionic, cationic, amphoterically charged or nonionic.

Cationic polymers are polymers containing groups which may be “temporarily” or “permanently” cationic in the main chain and/or side chain. According to the invention, “permanently cationic polymers” are polymers which contain a cationic group irrespective of the pH of the preparation. These are generally polymers which contain a quaternary nitrogen atom, for example in the form of an ammonium group. Preferred cationic groups are quaternary ammonium groups. Polymers where the quaternary ammonium group is attached by a C₁₋₄ hydrocarbon group to a polymer main chain made up of acrylic acid, methacrylic acid or derivatives thereof have proved to be particularly suitable.

Preferred cationic polymers are, for example,

the quaternized cellulose derivatives commercially available under the names of Celquat® and Polymer JR®. The compounds Celquat® H 100, Celquat® L 200 and Polymer JR® 400 are preferred quaternized cellulose derivatives,

the cationic alkyl polyglycosides according to DE-PS 44 13 868,

cationized honey, for example the commercial product Honeyquat® 50,

cationic guar derivatives such as, in particular, the products marketed under the names of Cosmedia® Guar and Jaguar®,

polysiloxanes containing quaternary groups such as, for example, the commercially available products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80),

polymeric dimethyl diallyl ammonium salts and copolymers thereof with esters and amides of acrylic acid and methacrylic acid. The products commercially available under the names of Merquat® 100 (poly(dimethyl diallylammonium chloride)) and Merquat® 550 (dimethyl diallylammonium chloride/acrylamide copolymer) are examples of such cationic polymers,

copolymers of vinyl pyrrolidone with quaternized derivatives of dialkylaminoacrylate and methacrylate such as, for example, vinyl pyrrolidone/dimethylaminoethyl methacrylate copolymers quaternized with diethyl sulfate. Such compounds are commercially available under the names of Gafquat® 734 and Gafquat® 755,

the vinyl pyrrolidone/vinyl imidazolinium methochloride copolymers commercially available under the names of Luviquat® FC 370, FC 550, FC 905 and HM 552,

quaternized polyvinyl alcohol;

and the polymers containing quaternary nitrogen atoms in the main polymer chain known under the names of Polyquaternium 2, Polyquaternium 17, Polyquaternium 18 and Polyquaternium 27.

Other suitable cationic polymers are the polymers known by the names of Polyquaternium 24 (commercial product: Quatrisoft® LM 200 for example). Also suitable for use in accordance with the invention are the vinyl pyrrolidone copolymers known by the commercial names of Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Other cationic polymers according to the invention are the so-called “temporarily cationic” polymers. These polymers typically contain an amino group which is present as a quaternary ammonium group, i.e. is cationic, at certain pH values. Preferred such polymers are, for example, chitosan and derivatives thereof which are freely available on the market, for example under the names of Hydagen® CMF, Hydagen® HCMF, Kytamer® PC and Chitolam® NB/101. Chitosans are deacetylated chitins which are commercially available in various degrees of deacetylation and various degrees of degradation (molecular weights). Their production is described, for example, in DE 44 40 625 A1 and in DE 195 03 465 A1.

Particularly suitable chitosans have a degree of deacetylation of at least 80% and a molecular weight of 5·10⁵ to 5·10⁶ (g/mol).

To produce preparations according to the invention, the chitosan has to be converted into the salt form. This can be done by dissolving the chitosan in dilute aqueous acids. Suitable acids are both mineral acids such as, for example, hydrochloric acid, sulfuric acid and phosphoric acid and organic acids, for example low molecular weight carboxylic acids, polycarboxylic acids and hydroxycarboxylic acids. Relatively high molecular weight alkyl sulfonic acids or alkyl sulfuric acids or organophosphorus acids may also be used providing they have the requisite physiological compatibility. Suitable acids for converting the chitosan into the salt form are, for example, acetic acid, glycolic acid, tartaric acid, malic acid, citric acid, lactic acid, 2-pyrrolidinone-5-carboxylic acid, benzoic acid or salicylic acid. Low molecular weight hydroxycarboxylic acids such as, for example, glycolic acid or lactic acid, are preferably used.

In addition, amphoteric polymers may be used as polymers for enhancing the effect of the active substance according to the invention. Amphoteric polymers are both polymers which contain both free amino groups and free —COOH or —SO₃H groups in the molecule and which are capable of forming inner salts and zwitterionic polymers which contain quaternary ammonium groups and —COO⁻ or —SO₃⁻ groups in the molecule and polymers which contain —COOH— or SO₃H groups and quaternary ammonium groups.

One example of an amphopolymer suitable for use in accordance with the invention is the acrylate resin commercially available as Amphomer® which is a copolymer of tert.butylaminoethyl methacrylate, N-(1,1,3,3-tetramethylbutyl)-acrylamide and two or more monomers from the group consisting of acrylic acid, methacrylic acid and simple esters thereof.

Another example of a polymer suitable for use in accordance with the invention is the Polyquaternium-22 obtainable under the name of Merquat® 280 which is a copolymer of dimethyl diallyl ammonium chloride and acrylic acid.

Other amphoteric polymers suitable for use in accordance with the invention are the compounds mentioned in GB-A-2,104,091, EP-A-0 047 714, EP-A-0 217 274, EP-A-0 283 817 and DE-A-28 17 369.

Preferred amphoteric polymers are polymers essentially consisting of

(a) monomers containing quaternary ammonium groups corresponding to general formula (VII):

R²²—CH═CR²³—CO—Z—(CₙH₂ₙ)—N⁽⁺⁾R²⁴R²⁵R²⁶A⁽⁻⁾ (VII)

in which R²² and R²³ independently of one another represent hydrogen or a methyl group and R²⁴, R²⁵ and R²⁶ independently of one another represent alkyl groups containing 1 to 4 carbon atoms, Z is an NH group or an oxygen atom, n is an integer of 2 to 5 and A⁽⁻⁾ is the anion of an organic or inorganic acid,

and

(b) monomeric carboxylic acids corresponding to general formula (VIII):

R²⁷—CH═CR²⁸—COOH (VIII)

in which R²⁷ and R²⁸ independently of one another are hydrogen or methyl groups.

According to the invention, these compounds may be used both directly and in the salt form obtained by neutralization of the polymers, for example with an alkali metal hydroxide. Particulars of the production of these polymers can be found in DE-A 39 29 973. Most particularly preferred polymers are those where type (a) monomers, in which R²⁴, R²⁵ and R²⁶ are methyl groups, Z is an NH group and A⁽⁻⁾ is a halide, methoxysulfate or ethoxysufate ion, are used; acrylamidopropyl trimethyl ammonium chloride is a particularly preferred monomer (a) Acrylic acid is preferably used as the monomer (b) for the polymers mentioned.

A preferred polymer of this type is, for example, the Polyquaternium-47 marketed by Calgon under the name of Merquat® 2001.

In a third variant, the preparations according to the invention may additionally contain nonionic care polymers.

Suitable nonionic polymers are, for example,

shellac,

siloxanes. These siloxanes may be both soluble and insoluble in water. Both volatile and nonvolatile siloxanes are suitable, nonvolatile siloxanes being understood to be those compounds which have a boiling point at normal pressure above 200° C. Preferred siloxanes are polydialkyl siloxanes such as, for example, polydimethyl siloxane, polyalkylaryl siloxanes such as, for example, polyphenyl methyl siloxane, ethoxylated polydialkyl siloxanes and polydialkyl siloxanes containing amine and/or hydroxy groups.

Glycosidically substituted silicones according to EP-B 1 0 612 759.

According to the invention, the preparations used may contain several, more particularly two, different care polymers with the same charge and/or an ionic polymer and an amphoteric polymer and/or nonionic polymer.

In the context of the present invention, care polymers are also understood to be special preparations of polymers, such as spherical polymer powders. There are various known processes for producing such microspheres from various monomers, for example by special polymerization processes or by dissolving the polymer in a solvent and spraying the solution into a medium in which the solvent is able to evaporate or diffuse out of the particles. One such process is known, for example, from EP-B1-0 466 986. Suitable polymers are, for example, polycarbonates, polyurethanes, polyacrylates, polyolefins, polyesters or polyamides. Spherical polymer powders with a primary particle diameter below 1 μm are particularly suitable. Corresponding products based on a polymethacrylate copolymer are commercially available, for example, under the registered name of Polytrap® Q5-6603 (Dow Corning). Other polymer powders, for example based on polyamides (Nylon 6, Nylon 12), are commercially available in a particle size of 2 to 10 μm (90%) and a specific surface of ca. 10 m²/g under the name of Orgasol® 2202 DU Nat Cos (Atochem S.A., Paris). Other spherical polymer powders suitable for the purposes of the invention are, for example, the polymethacrylates (Micropearl M) from SEPPIC or (Plastic Powder A) from NIKKOL, the styrene/divinylbenzene copolymers (Plastic Powder FP) from NIKKOL, the polyethylene and polypropylene powders (ACCUREL EP 400) from AKZO and even silicone polymers (Silicone Powder X2-1605) from Dow Corning or spherical cellulose powder.

The care polymers are preferably present in the preparations used in accordance with the invention in quantities of 0.01 to 10% by weight, based on the preparation as a whole. Quantities of 0.1 to 5% by weight and more particularly 0.1 to 2% by weight are particularly preferred.

Other Constituents

To produce the colorants according to the invention, the oxidation dye precursors are incorporated in a suitable water-containing carrier. For coloring hair, such carriers are, for example, creams, emulsions, gels or even surfactant-containing foaming solutions, for example shampoos, foam aerosols or other formulations suitable for application to the hair.

The colorants according to the invention may also contain any of the known active substances, additives and auxiliaries typical of such formulations. In many cases, the colorants contain at least one surfactant, both anionic and zwitterionic, ampholytic, nonionic and cationic surfactants being suitable in principle. If necessary, the expert can check the various surfactants for possible effects on the activity of the enzyme system according to the invention by carrying out simple preliminary tests.

In a preferred embodiment of the present invention, a combination of anionic and nonionic surfactants or a combination of anionic and amphoteric surfactants is used in the preparations for coloring keratinous fibers.

In individual cases, however, it has been found to be of advantage to select the surfactants from amphoteric or nonionic surfactants because they generally have relatively effect on the coloring process according to the invention.

Suitable anionic surfactants for the preparations according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide and hydroxyl groups may also be present in the molecule. The following are examples of suitable anionic surfactants—in the form of the sodium, potassium and ammonium salts and the mono-, di- and trialkanol-ammonium salts containing 2 or 3 carbon atoms in the alkanol group:

linear fatty acids containing 10 to 22 carbon atoms (soaps),

ether carboxylic acids corresponding to the formula R—O—(CH₂—CH₂O)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms,

protein/acid concentrates,

coconut monoglyceride sulfates,

amide ether sulfates.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

Nonionic surfactants contain, for example, a polyol group, a polyalkylene glycol ether group or a combination of polyol and polyglycol ether groups as the hydrophilic group. Examples of such compounds are

products of the addition of 2 to 30 mol ethylene oxide and/or 0 to 5 mol propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of products of the addition of 1 to 30 mol ethylene oxide onto glycerol,

C₈₋₂₂ alkyl mono- and oligoglycosides and ethoxylated analogs thereof,

products of the addition of 5 to 60 mol ethylene oxide onto castor oil and hydrogenated castor oil,

Zwitterionic surfactants may also be used, particularly as co-surfactants. In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, cocoacylaminopropyl dimethyl ammonium glycinate and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the INCI name of Cocamidopropyl Betaine.

Also suitable, particularly as co-surfactants, are ampholytic surfactants. Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

Examples of the cationic surfactants suitable for use in the hair treatment preparations according to the invention are, in particular, quaternary ammonium compounds, esterquats and amidoamines.

Preferred quaternary ammonium compounds are ammonium halides, more particularly chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride and the imidazolium compounds known under the INCI names of Quaternium-27 and Quaternium-83. The long alkyl chains of the above-mentioned surfactants preferably contain 10 to 18 carbon atoms.

Esterquats are known substances which contain both at least one ester function and at least one quaternary ammonium group as structural element. Preferred esterquats are quaternized ester salts of fatty acids with triethanolamine, quaternized ester salts of fatty acids with diethanol alkylamines and quaternized ester salts of fatty acids with 1,2-dihydroxypropyl dialkylamines. Such products are marketed, for example, under the names of Stepantex®, Dehyquart® and Armocare®. The products Armocare® VGH-70, an N,N-bis-(2-palmitoyloxyethyl)-dimethyl ammonium chloride, and Dehyquart® F-75 and Dehyquart® AU-35 are examples of such esterquats.

The alkyl amidoamines are normally prepared by amidation of natural or synthetic fatty acids and fatty acid cuts with dialkyl aminoamines. A compound from this group particularly suitable for the purposes of the invention is the stearamidopropyl dimethylamine obtainable under the name of Tegoamid® S 18.

Other cationic surfactants suitable for use in accordance with the invention are the quaternized protein hydrolyzates.

Also suitable for use in accordance with the invention are cationic silicone oils such as, for example, polysiloxanes containing quaternary groups such as, for example, the commercially available products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

One example of a quaternary sugar derivative suitable for use as a cationic surfactant is the commercially available product Glucquat®100 (INCI name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

In addition, the preparations according to the invention preferably contain at least one alkalizing agent. Preferred alkalizing agents are ammonia, monoethanolamine, isopropylamine, isopropanolamine, 2-amino-2-methylpropan-1-ol, an alkali metal hydroxide, more especially sodium hydroxide or potassium hydroxide, and more particularly arginine, lysine and histidine. Particularly preferred colorants according to the invention contain monoethanolamine, 2-amino-2-methylpropan-1-ol, an alkali metal hydroxide, more especially sodium hydroxide or potassium hydroxide, and more particularly arginine, lysine and histidine.

In addition, the colorants according to the invention may preferably contain a conditioning agent selected from the group consisting of cationic surfactants, alkyl amidoamines, paraffin oils, vegetable oils and synthetic oils. With regard to the cationic surfactants, reference is made to the foregoing observations.

Other suitable conditioning agents are silicone oils, more particularly dialkyl and alkylaryl siloxanes, such as for example dimethyl polysiloxane and methylphenyl polysiloxane, and alkoxylated and quaternized analogs thereof. Examples of such silicones are the products marketed by Dow Corning under the names of DC 190, DC 200, DC 344, DC 345 and DC 1401 and the products Q2-7224 (manufacturer: Dow Corning; a stabilized trimethyl silyl amodimethicone), Dow Corning® 929 Emulsion (containing a hydroxylamino-modified silicone which is also known as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, Quaternium-80).

Other suitable conditioning agents are paraffin oils, synthetically produced oligomeric alkenes and vegetable oils, such as jojoba oil, sunflower oil, orange oil, almond oil, wheatgerm oil and peach kernel oil.

Phospholipids, for example soya lecithin, egg lecithin and kephalins, are also suitable hair-conditioning compounds.

In addition, the preparations used in accordance with the invention preferably contain at least one oil component.

Oil components suitable for the purposes of the invention are, in principle, any water-insoluble oils and fatty compounds and mixtures thereof with solid paraffins and waxes. According to the invention, water-insoluble substances are defined as substances of which less than 0.1% by weight dissolves in water at 20° C. The melting point of the individual oil or fatty components is preferably below about 40° C. Oil and fatty components which are liquid at room temperature, i.e. below 25° C., can be particularly preferred for the purposes of the invention. However, where several oil and fatty components and optionally solid paraffins and waxes are used, it is generally sufficient if the mixture of the oil and fatty components and optionally paraffins and waxes satisfies these requirements.

A preferred group of oil components are vegetable oils. Examples of such oils are sunflower oil, olive oil, soya oil, rapeseed oil, almond oil, jojoba oil, orange oil, wheatgerm oil, peach kernel oil and the liquid fractions of coconut oil.

However, other triglyceride oils, such as the liquid fractions of bovine tallow, and synthetic triglyceride oils are also suitable.

Another group of compounds particularly preferred for use as oil components in accordance with the invention are liquid paraffin oils and synthetic hydrocarbons and di-n-alkyl ethers containing a total of 12 to 36 carbon atoms and, more particularly, 12 to 24 carbon atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and ditert.butyl ether, diisopentyl ether, di-3-ethyldecyl ether, tert.butyl-n-octyl ether, isopentyl-n-octyl ether and 2-methylpentyl-n-octyl ether. The compounds 1,3-di-(2-ethylhexyl)-cyclohexane and di-n-octyl ether obtainable as commercial products (Cetiol® S and Cetiol® OE, respectively) can be preferred.

Other oil components suitable for use in accordance with the invention are fatty acid and fatty alcohol esters. The monoesters of fatty acids with alcohols containing 3 to 24 carbon atoms are preferred. This group of substances are products of the esterification of fatty acids containing 8 to 24 carbon atoms such as, for example, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and the technical mixtures thereof obtained, for example, in the pressure hydrolysis of natural fats and oils, in the reduction of aldehydes from Roelen's oxosynthesis or in the dimerization of unsaturated fatty acids with alcohols such as, for example, isopropyl alcohol, caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxosynthesis and as monomer fraction in the dimerization of unsaturated fatty alcohols. According to the invention, isopropyl myristate, isononanoic acid C₁₆₋₁₈ alkyl ester (Cetiol® SN), stearic acid-2-ethylhexyl ester (Cetiol® 868), cetyl oleate, glycerol tricaprylate, cocofatty alcohol caprate/caprylate and n-butyl stearate are particularly preferred.

Other oil components suitable for use in accordance with the invention are dicarboxylic acid esters, such as di-n-butyl adipate, di-(2ethylhexyl)-adipate, di-(2-ethylhexyl)-succinate and diisotridecyl acelate, and diol esters, such as ethylene glycol dioleate, ethylene glycol diisotridecanoate, propylene glycol di-(2-ethylhexanoate), propylene diisostearate, propylene glycol dipelargonate, butanediol diisostearate and neopentyl glycol dicaprylate, and also complex esters, for example diacetyl glycerol monostearate.

Finally, fatty alcohols containing 8 to 22 carbon atoms may also be used as oil components in accordance with the invention. The fatty alcohols may be saturated or unsaturated and linear or branched. Examples of fatty alcohols suitable for use in accordance with the invention are decanol, octanol, octenol, dodecenol, decenol, octadienol, dodecadienol, decadienol, oleyl alcohol, erucyl alcohol, ricinolyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol and Guerbet alcohols thereof (this list is purely exemplary and is not intended to limit the invention in any way). However, the fatty alcohols emanate from preferably natural fatty acids, normally being obtained from the esters of the fatty acids by reduction. According to the invention, it is also possible to use the fatty alcohol cuts which are produced by reduction of naturally occurring triglycerides, such as bovine tallow, palm oil, peanut oil, rapeseed oil, cottonseed oil, soybean oil, sunflower oil and linseed oil, or fatty acid esters formed from the transesterification products thereof with corresponding alcohols and which therefore represent a mixture of different fatty alcohols.

The oil components are used in the colorants according to the invention in quantities of preferably 0.05 to 10% by weight and more particularly 0.1 to 2% by weight.

Other active substances, auxiliaries and additives are, for example,

structurants, such as maleic acid and lactic acid,

protein hydrolyzates, more particularly elastin, collagen, keratin, milk protein, soya protein and wheat protein hydrolyzates, condensation products thereof with fatty acids and quaternized protein hydrolyzates,

perfume oils, dimethyl isosorbide and cyclodextrins,

solvents and solubilizers, such as ethanol, isopropanol, ethylene glycol, propylene glycol, glycerol and diethylene glycol,

fiber structure improvers, more particularly mono-, di- and oligosaccharides such as, for example, glucose, galactose, fructose and lactose,

quaternized amines, such as methyl-1-alkylamidoethyl-2-alkylimidazolinium methosulfate,

defoamers, such as silicones,

dyes for coloring the composition,

antidandruff agents, such as Piroctone Olamine, Zinc Omadine and Climbazol,

sun protection factors, more particularly derivatized benzophenones, cinnamic acid derivatives and triazines,

substances for adjusting the pH value, such as for example the usual acids, more particularly edible acids and bases,

active principles, such as allantoin, pyrrolidone carboxylic acids and salts thereof and bisabolol,

vitamins, provitamins and vitamin precursors, more particularly those of groups A, B₃, B₅, B₆, C, E, F and H,

plant extracts, such as the extracts of green tea, oak bark, stinging nettle, hamamelis, hops, camomile, burdock root, horse willow, hawthorn, lime blossom, almond, aloe vera, pine needle, horse chestnut, sandalwood, juniper, coconut, mango, apricot, lemon, wheat, kiwi, melon, orange, grapefruit, sage, rosemary, birch, mallow, lady's smock, creeping thyme, yarrow, thyme, balm, restharrow, coltsfoot, hibiscus, meristem, ginseng and ginger root,

cholesterol,

consistency factors, such as sugar esters, polyol esters or polyol alkyl ethers,

fats and waxes, such as spermaceti, beeswax, montan wax and paraffins,

fatty acid alkanolamides,

complexing agents, such as EDTA, NTA, β-alanine diacetic acid and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex, styrene/PVP and styrene/acrylamide copolymers,

pearlizers, such as ethylene glycol mono- and distearate and PEG-3-distearate,

pigments,

stabilizers for hydrogen peroxide and other oxidizing agents,

propellents, such as propane/butane mixtures, N₂O, dimethyl ether, CO₂ and air,

antioxidants and

preservatives.

Information on other optional components and the quantities in which they are used can be found in the reference books known to the expert, for example Kh. Schrader, Grundlagen und Rezepturen der Kosmetika, 2nd Edition, Hüthig Buch Verlag, Heidelberg, 1989.

The enzyme preparation is preferably mixed with the preparation of the oxidation dye precursors immediately before coloring of the hair. The ready-to-use hair coloring preparation formed should have a pH value preferably in the range from 4 to 10, more preferably in the range from 6 to 9 and most preferably in the range from 7 to 8.5. In addition, low-viscosity preparations with a viscosity of less than 3,000 mPas (Brookfield, spindle 4, 20 r.p.m., 20° C.) have proved to be particularly advantageous. The application temperatures may be in the range from 10 to 50° C. and are preferably in the range from 20 to 35° C. After a contact time of about 30 minutes, the hair colorant is removed from the hair to be colored by rinsing. There is no need for the hair to be washed with a shampoo where a carrier of high surfactant content, for example a coloring shampoo, has been used.

In order further to increase the coloring performance of the preparations according to the invention, it can be of advantage in accordance with the invention to subject the fibers after the treatment with the preparation according to the invention to an aftertreatment with a solution containing small quantities of hydrogen peroxide. This solution may be formulated, for example, as a shampoo or conditioner. Such aftertreatment preparations normally contain hydrogen peroxide in concentrations of 0.01 to 0.5% by weight.

In another preferred embodiment of the present invention, the preparations and especially the separately prepared enzyme preparation may be formulated without antioxidants and/or complexing agents because these can block the effect of the enzymes.

In a second embodiment, the present invention relates to a process for coloring keratinous fibers in which a preparation according to the invention is applied to the fibers and, after a contact time, is rinsed out again.

In a third embodiment, the present invention relates to a process for coloring keratinous fibers in which, in a first step, a preparation containing components (A), (C) and (D) is applied to the fibers, the fibers are optionally rinsed after a contact time and, in a second step, a preparation containing component (B) is applied to the fibers. Another process which has also proved to be suitable for the purposes of the invention is characterized in that, in a first step, the fibers are treated with a preparation containing component (B), the fibers are optionally rinsed after a contact time and a preparation containing components (A), (C) and (D) is then applied.

In a fourth embodiment, the present invention relates to the use of the preparations according to the invention for the oxidative coloring of keratinous fibers.

In a fifth embodiment, the present invention relates to a kit for coloring keratinous fibers which comprises at least a first preparation containing

(A) at least one dye precursor,

(C) at least one sugar surfactant selected from the group consisting of

alkyl and alkenyl oligoglycosides and

fatty acid-N-alkyl polyhydroxyalkyl amides and

(D) at least one reducing agent in a cosmetically acceptable carrier and a second preparation containing

(B) at least one enzyme capable of catalyzing the oxidation of the dye precursors

in a cosmetically acceptable carrier.

The following Examples are intended to illustrate the invention.

### EXAMPLES

The quantities in the following Examples are percentages by weight unless otherwise indicated.

| I) | Composition of the coloring gels | |
|---|---|---|
| | Coloring gel 1: | |
| | Natrosol ® 250 HR¹ | 2.0 |
| | Eumulgin ® B2² | 0.5 |
| | Plantacare ® 1200 UP³ | 0.5 |
| | Lamesoft ® PO65⁴ | 0.2 |
| | trisodium citrate dihydrate | 0.5 |
| | 2,4,5,6-tetraaminopyrimidine sulfate | 0.1 |
| | p-toluylenediamine sulfate | 0.1 |
| | 4-amino-3-methylphenol | 0.25 |
| | m-aminophenol | 0.1 |
| | 4-chlororesorcinol | 0.12 |
| | 2-amino-3-hydroxypyridine | 0.13 |
| | monoethanolamine | to pH 7 |
| | water | to 100 |
| | Coloring gel 2: | |
| | Natrosol ® 250 HR | 2.0 |
| | Plantacare ® 1200 UP | 0.5 |
| | Lamesoft ® PO65 | 0.2 |
| | Polymer W 37194⁵ | 0.1 |
| | trisodium citrate dihydrate | 0.5 |
| | 2,4,5,6-tetraaminopyrimidine sulfate | 0.1 |
| | 1-(β-hydroxyethyl)-p-phenylenediamine sulfate | 0.1 |
| | 4-amino-3-methylphenol | 0.2 |
| | m-aminophenol | 0.1 |
| | 4-chlororesorcinol | 0.1 |
| | 2-amino-3-hydroxypyridine | 0.13 |
| | Rodol 9R Base⁶ | 0.1 |
| | monoethanolamine | to pH 7 |
| | water | to 100 |
| | Coloring gel 3: | |
| | Natrosol ® 250 HR | 2.0 |
| | Eumulgin ® B2 | 0.5 |
| | Plantacare ® 1200 UP | 1.0 |
| | Lamesoft ® PO65 | 0.5 |
| | N-acetyl-L-cysteine | 0.15 |
| | 2,4,5,6-tetraaminopyrimidine sulfate | 1.5 |
| | p-phenylenediamine sulfate | 0.2 |
| | 4-amino-2-aminomethyl phenol dihydrochloride | 0.05 |
| | 2-methyl resorcinol | 1.0 |
| | 1-(β-hydroxyethylamino)-4-methyl-2-nitrobenzene | 0.1 |
| | L-arginine | 1.0 |
| | monoethanolamine | to pH 7 |
| | water | to 100 |
| | Coloring gel 4: | |
| | Natrosol ® 250 HR | 2.0 |
| | Eumulgin ® B2 | 0.5 |
| | Plantacare ® 1200 UP | 0.5 |
| | Lamesoft ® PO65 | 0.2 |
| | trisodium citrate dihydrate | 0.5 |
| | 2,4,5,6-tetraaminopyrimidine sulfate | 0.2 |
| | 4,5-diamino-1-(β-hydroxyethyl)-pyrazole | 0.8 |
| | 4-amino-3-methylphenol | 0.4 |
| | 2-methyl resorcinol | 0.2 |
| | 2,7-dihydroxynaphthalene | 0.6 |
| | 4-chlororesorcinol | 0.2 |
| | lysine | 0.2 |
| | isopropanolamine | to pH 7 |
| | water | to 100 |
| | Coloring gel 5: | |
| | Natrosol ® 250 HR | 2.0 |
| | Eumulgin ® B2 | 0.5 |
| | Plantacare ® 1200 UP | 0.5 |
| | Lamesoft ® PO65 | 0.2 |
| | N-acetyl-L-cysteine | 0.2 |
| | p-toluylenediamine sulfate | 1.1 |
| | p-aminophenol | 0.1 |
| | 2-methyl resorcinol | 0.1 |
| | 2,7-dihydroxynaphthaline | 0.3 |
| | 5,6-dihydroxyindoline hydrobromide | 0.2 |
| | m-aminophenol | 0.06 |
| | 2-methylamino-3-amino-6-methoxypyridine | 0.02 |
| | 2-amino-3-hydroxypyridine | 0.01 |
| | resorcinol | 0.2 |
| | monoethanolamine | to pH 7 |
| | water | to 100 |
| | Coloring gel 6: | |
| | Natrosol ® 250 HR | 2.0 |
| | Eumulgin ® B2 | 0.5 |
| | Plantacare ® 1200 UP | 1.5 |
| | trisodium citrate dihydrate | 0.2 |
| | N-acetyl-L-cysteine | 0.2 |
| | p-toluylenediamine sulfate | 0.6 |
| | bis-(2-hydroxy-5-aminophenyl)-methane | 0.03 |
| | 2-methyl resorcinol | 0.07 |
| | m-aminophenol | 0.03 |
| | 4-chlororesorcinol | 0.18 |
| | resorcinol | 0.1 |
| | histidine | 0.5 |
| | monoethanolamine | to pH 7 |
| | water | to 100 |
| | Coloring gel 7: | |
| | Carbopol ® 934⁷ | 1.5 |
| | Eumulgin ® B2 | 0.5 |
| | Plantacare ® 1200 UP | 1.5 |
| | Polymer JR ® 400⁸ | 0.5 |
| | trisodium citrate dihydrate | 0.5 |
| | 2,4,5,6-tetraaminopyrimidine sulfate | 0.2 |
| | p-toluylenediamine sulfate | 0.9 |
| | 4-amino-2-aminomethylphenol dihydrochloride | 0.4 |
| | 2-methyl resorcinol | 0.25 |
| | 2,7-dihydroxynaphthalene | 0.6 |
| | 4-chlororesorcinol | 0.24 |
| | 5,6-dihydroxyindoline hydrobromide | 0.2 |
| | L-arginine | 1.0 |
| | isopropanolamine | to pH 7 |
| | water | to 100 |
| | Coloring gel 8: | |
| | Carbopol ® 934 | 1.0 |
| | Eumulgin ® B2 | 0.2 |
| | Eumulgin ® B1⁹ | 0.2 |
| | Plantacare ® 1200 UP | 0.5 |
| | Lamesoft ® PO65 | 0.2 |
| | tripotassium citrate dihydrate | 0.5 |
| | 2,4,5,6-tetraaminopyrimidine sulfate | 0.7 |
| | p-toluylenediamine sulfate | 0.4 |
| | 2-(β-hydroxyethyl)-p-phenylenediamine sulfate | 0.1 |
| | 3-methyl-4-aminophenol | 0.5 |
| | 2-methyl resorcinol | 0.5 |
| | 2,7-dihydroxynaphthalene | 0.16 |
| | 4-chlororesorcinol | 0.1 |
| | 2-amino-3-hydroxypyridine | 0.5 |
| | Rodol 9R Base | 0.1 |
| | histidine | 0.2 |
| | monoethanolamine | to pH 7 |
| | water | to 100 |
| | Coloring gel 9: | |
| | Natrosol ® 250 HR | 2.0 |
| | Eumulgin ® B2 | 0.2 |
| | Eumulgin ® B1 | 0.2 |
| | Plantacare ® 1200 UP | 1.5 |
| | Lamesoft ® PO65 | 0.2 |
| | Merquat ® 280¹⁰ | 0.8 |
| | trisodium citrate dihydrate | 1.0 |
| | 2,4,5,6-tetraaminopyrimidine sulfate | 0.6 |
| | p-toluylenediamine sulfate | 0.02 |
| | 3-methyl-4-aminophenol | 0.6 |
| | 4-chlororesorcinol | 0.03 |
| | 2-amino-3-hydroxypyridine | 0.5 |
| | 1-(β-hydroxyethylamino)-4-methyl-2-nitrobenzene | 0.1 |
| | isopropanolamine | to pH 7 |
| | water | to 100 |
| | Coloring gel 10: | |
| | Aculyn ® 22¹¹ | 2.0 |
| | Eumulgin ® B2 | 0.5 |
| | Plantacare ® 1200 UP | 0.5 |
| | Lamesoft ® PO65 | 0.2 |
| | trisodium citrate dihydrate | 0.5 |
| | tripotassium citrate dihydrate | 0.5 |
| | 2,4,5,6-tetraaminopyrimidine sulfate | 0.6 |
| | p-toluylenediamine sulfate | 0.27 |
| | 2,6-di-(β-hydroxyethylamino)-toluene | 0.4 |
| | 3,5-diamino-2,6-dimethoxypyridine | 0.5 |
| | sodium hydroxide | to pH 7 |
| | water | to 100 |
| | Coloring gel 11: | |
| | Natrosol ® 250 HR | 3.0 |
| | Eumulgin ® B2 | 0.5 |
| | Plantacare ® 1200 UP | 1.5 |
| | Lamesoft ® PO65 | 0.2 |
| | trisodium citrate dihydrate | 0.5 |
| | p-toluylenediamine sulfate | 1.1 |
| | 1,3-bis-(2′,4′-diaminophenoxy)-propane hydrochloride | 0.4 |
| | m-aminophenol | 0.1 |
| | 2-methylamino-3-amino-6-methoxypyridine | 0.2 |
| | 2-amino-4-(β-hydroxyethylamino)-anisole sulfate | 0.2 |
| | monoethanolamine | to pH 7 |
| | water | to 100 |

¹hydroxyethyl cellulose (INCI name: Hydroxyethylcellulose) (Hercules)
²cetylstearyl alcohol containing ca. 20 EO units (INCI name: Ceteareth-20) (Cognis)
³C₁₂₋₁₆ alkyl-1,4-glucoside (ca. 50 to 53% active substance) INCI name: Lauryl Glucoside) (Cognis)
⁴alkyl polyglucoside/oleic acid monoglyceride mixture (ca. 32 to 35% water content, INCI name: Glucoside, Glyceryl Oleate, Aqua (Water)) (Cognis)
⁵acrylic acid sodium salt/acrylamidopropyl trimethyl ammonium chloride copolymer preserved with Phenonip (ca. 19-21% active substance, INCI name: Acrylamidopropyl-Trimonium Chloride/Acrylates Copolymer) (Stockhausen)
⁶2-amino-6-chloro-4-nitrophenol
⁷carboxyvinyl polymer (INCI name: Carbomer) (Goodrich)
⁸quaternized hydroxyethyl cellulose (INCI name: Polyquaternium-10) (Amerchol)
⁹cetylstearyl alcohol containing ca. 12 EO units (INCI name: Ceteareth-12) (Cognis)
¹⁰dimethyl diallyl ammonium chloride/acrylic acid copolymer (ca. 35% active substance, INCI name: Polyquaternium-22) (Chemviron)
¹¹methacrylic acid(stearyl alcohol + 20 EO ester)/acrylic acid copolymer (ca. 30% active substance, INCI name: Acrylates/Steareth-20 Methacrylate Copolymer) (Rohm & Haas)

II) Enzymes

a) Laccase

A laccase from Rhus vernificera (Sigma) was used. According to the manufacturer, the activity of the laccase is so defined that one unit [1U] corresponds to the quantity of laccase which produces a ΔA₅₃₀ of 0.001 per minute at a pH value of 6.5 and a temperature of 30° C. in 3 ml of a reaction solution containing syringaldazin as substrate. The laccase was used in a quantity which has an activity of 280,000 U under the conditions mentioned above.

b) Ascorbate Oxidase

An ascorbate oxidase from Curcurbita species (Roche Diagnostics) was used. The activity of the ascorbate oxidase is so defined that one unit [1 U] corresponds to the quantity of ascorbate oxidase which catalyzes the oxidation of 1 μmol L-ascorbic acid in 1 minute at pH 5.6/25° C. in a phosphate buffer solution (KH₂PO₄, 0.1 mol/l; Na₂HPO₄, 4 mmol/l; EDTA, 0.5 mmol/l), corresponding to the definition of the Roche Diagnostics products. Detection is by spectroscope and is based on the reduction in absorption at 245 nm. The ascorbate oxidase was used in a quantity which has an activity of 100,000 U under the conditions mentioned above.

c) Bilirubin Oxidase

A bilirubin oxidase from Myrothecium verrucaria (Sigma) was used. The activity of the bilirubin oxidase is so defined that one unit [1 U] corresponds to the quantity of bilirubin oxidase which catalyzes the oxidation of 1 μmol bilirubin in one minute at pH 8.4/37° C. The bilirubin oxidase was used in a quantity which has an activity of 1,000 U under the conditions mentioned above.

d) Uricase

A uricase from Arthrobacter globiformis (Sigma) was used. The activity of the uricase is so defined that one unit [1U] corresponds to the quantity of uricase which catalyzes the conversion of 1 μmol uric acid into allantoin in one minute at pH 8.5/25° C.

The uricase was used in a quantity which has an activity of 25,000 U under the conditions mentioned above.

III) Coloring

Enzymes a to c

60 g of the particular coloring gel 1 to 11 were mixed with 10 ml of a solution buffered to pH 7 (by KHPO4/KH₂PO₄) which contains enzymes a-c in the activities mentioned in II. The mixture was applied to human hair (Kerling, natural white, 4 g colorant per 0.5 g hair), worked in and left for 30 mins. at room temperature. The hair was then rinsed and dried.

Enzyme d 0.35 g uric acid and the quantity of uricase defined in II were added to quantities of 60 g of coloring gels 1 to 11. After making up to 70 g with twice-distilled water, coloring was carried out by the method described for enzymes a to c.

The coloring results obtained are set out in the following Table.

| | Color | | | |
|---|---|---|---|---|
| | | Ascorbate | | |
| | Laccase | oxidase | Bilirubin oxidase | Uricase |
| Coloring gel 1 | Deer brown | Autumn brown | Autumn brown | Light gold- |
| | | | | brown |
| Coloring gel 2 | Autumn red | Red-brown | Autumn red | Mahogany |
| Coloring gel 3 | Garnet red | Garnet red | Garnet red | Garnet red |
| Coloring gel 4 | Mid-brown | Light brown | Mid-brown | Chocolate |
| | | | | brown |
| Coloring gel 5 | Dark brown | Mid-dark brown | Mid-dark brown | Brown |
| Coloring gel 6 | Light gold-brown | Gold-brown | Gold-brown | Mid-brown |
| Coloring gel 7 | Hazelnut brown | Yellow-brown | Yellow-brown | Mid-brown-gold |
| Coloring gel 8 | Red-copper | Copper-red | Copper-gold | Copper |
| Coloring gel 9 | Copper | Copper-brown | Copper-gold | Copper-gold |
| Coloring gel 10 | Dark violet | Dark violet | Dark violet | Violet |
| Coloring gel 11 | Black | Black | Black | Dark grey |

1. A preparation for coloring keratinous fibers which contains
(A) at least one dye precursor,
(B) at least one enzyme capable of catalyzing the oxidation of the dye precursors,
(C) at least one sugar surfactant selected from the group consisting of
alkyl and alkenyl oligoglycosides and
fatty acid-N-alkyl polyhydroxyalkyl amides and
(D) at least one reducing agent
in a cosmetically acceptable carrier.

2. A preparation as claimed in claim 1, characterized in that it contains at least one primary intermediate as the dye precursor (A).

3. A preparation as claimed in claim 1 or 2, characterized in that it contains at least one indole and/or indoline derivative as the dye precursor (A).

4. A preparation as claimed in any of claims 1 to 3, characterized in that it contains at least one secondary intermediate.

5. A preparation as claimed in any of claims 1 to 4, characterized in that it additionally contains at least one substantive dye.

6. A preparation as claimed in any of claims 1 to 5, characterized in that it contains an alkali metal citrate salt, more particularly sodium citrate, and/or N-acetyl-L-cysteine as the reducing agent.

7. A preparation as claimed in claim 6, characterized in that it contains N-acetyl-L-cysteine as the reducing agent.

8. A preparation as claimed in any of claims 1 to 7, characterized in that it additionally contains at least one fatty acid partial glyceride.

9. A preparation as claimed in any of claims 1 to 8, characterized in that it additionally contains at least one thickener.

10. A preparation as claimed in claim 9, characterized in that the thickener is an anionic or nonionic polymer.

11. A preparation as claimed in claim 9 or 10, characterized in that it contains at least one cellulose and/or a cellulose derivative as thickener.

12. A preparation as claimed in any of claims 1 to 11, characterized in that it additionally contains a care polymer.

13. A preparation as claimed in any of claims 1 to 12, characterized in that it contains at least one other surfactant.

14. A preparation as claimed in claim 13, characterized in that it contains a nonionic or amphoteric surfactant.

15. A preparation as claimed in any of claims 1 to 14, characterized in that it contains at least one alkalizing agent.

16. A preparation as claimed in claim 15, characterized in that it contains monoethanolamine, isopropyl amine, isopropanolamine, 2-amino-2-methylpropan-1-ol, arginine, histidine or an alkali metal hydroxide, more particularly sodium hydroxide or potassium hydroxide, as the alkalizing agent.

17. A preparation as claimed in any of claims 1 to 16, characterized in that it has a pH value of 4 to 10, preferably 6 to 9 and more particularly 7 to 8.5.

18. A preparation as claimed in any of claims 1 to 17, characterized in that it is a low-viscosity preparation with a viscosity of less than 3,000 mPas (Brookfield, spindle 4, 20 r.p.m., 20° C.).

19. A process for coloring keratinous fibers in which the preparation claimed in any of claims 1 to 18 is applied to the fibers and, after a contact time, is rinsed off again.

20. A process for coloring keratinous fibers in which, in a first step, a preparation containing components (A), (C) and (D) is applied to the fibers, the fibers are optionally rinsed after a contact time and, in a second step, a preparation containing component (B) is applied to the fibers.

21. The use of any of the preparations claimed in claims 1 to 18 for the oxidative coloring of keratinous fibers.

22. A kit for coloring keratinous fibers which comprises at least a first preparation containing
(A) at least one dye precursor,
(C) at least one sugar surfactant selected from the group consisting of
alkyl and alkenyl oligoglycosides and
fatty acid-N-alkyl polyhydroxyalkyl amides and
(D) at least one reducing agent in a cosmetically acceptable carrier and a second preparation containing
(B) at least one enzyme capable of catalyzing the oxidation of the dye precursors
in a cosmetically acceptable carrier.
